SEQUENCE LISTING (1 sequence)

SEQ ID NO: 1 - DNA, 90 bp, Hordeum vulgare
tgcttacagtccgctgcaaaaggggttaatccaagtctagcctctggccttcctggaaagtgcggtgttagcatcccctatcccatctcc

(19) United States
(12) Patent Application Publication
(10) Pub. No.: US 2006/0263813 A1
(43) Pub. Date: Nov. 23, 2006

(54) Methods of monitoring functional status of transplants using gene panels

(75) Inventors: Steven Rosenberg, Oakland, CA (US); Preeti G. Lal, Santa Clara, CA (US); Kirk E. Fry, Palo Alto, CA (US); Tod M. Klingler, San Carlos, CA (US); Dirk Walther, Berlin (DE); Robert N. Woodward, Pleasanton, CA (US)

Correspondence Address: MORRISON & FOERSTER LLP, 425 MARKET STREET, SAN FRANCISCO, CA, 94105-2482, US

(73) Assignee: EXPRESSION DIAGNOSTICS, INC., South San Francisco, CA (US)

(21) Appl. No.: 11/433,191

(22) Filed: May 11, 2006

Related U.S. Application Data
(60) Provisional application No. 60/680,442, filed on May 11, 2005.

(51) Int. Cl. C12Q 1/68

(52) U.S. Cl. 435/6

(57) ABSTRACT

Methods useful in monitoring the functional status of a transplant in a patient by detecting the expression levels of gene panels are described herein. Algorithms for analysis of the expression for monitoring the functional status of transplants are also described.

### RELATED APPLICATION

This non-provisional application claims the benefit of U.S. Patent Application 60/680,442, filed May 11, 2005, which is hereby incorporated by reference in its entirety.

### FIELD OF THE INVENTION

This invention is in the field of expression profiling for monitoring the functional status of transplants. The invention may be particularly applied to heart and lung transplantation.

### BACKGROUND OF THE INVENTION

Transplant of an organ or tissue from one individual to another has become increasingly routine as newer and more sophisticated immunosuppression regimens have been developed to prevent and treat rejection of the transplanted organ or tissue. An essential component of such immunosuppression regimens is the monitoring of the recipient for the status of the transplant, i.e., is the recipient rejecting the organ or tissue. The current method of determining whether a recipient of a transplanted organ is rejecting that organ varies depending upon the organ. Heart transplant, by way of example, involves taking a biopsy of the transplanted organ. The biopsy is then examined for signs of rejection and rated on a four point scale. However, this method is invasive, expensive, painful, and associated with significant risk and has inadequate sensitivity for focal rejection. Other methods include detecting breakdown products specific to dysfunction of the transplanted organ. Therefore, there is a need for reliable alternative methods of diagnosing and monitoring transplant rejection that are less invasive and can provide better future prediction of graft dysfunction rather than merely detecting the actual dysfunction. One promising new technology being applied to diagnosing and monitoring transplant rejection is gene expression profiling. In particular, being able to diagnose and monitor transplant patients by gene expression profiling of peripheral blood would be particularly advantageous as it is relatively non-invasive to take a blood sample from a patient.

Recently, several genes have been identified that may be used to monitor transplant rejection. PCT application WO 02/057414 “LEUKOCYTE EXPRESSION PROFILING” to Wohlgemuth identifies a set of differentially expressed nucleotides that may be used to monitor transplant rejection. While the expression of individual genes may be measured to monitor transplant rejection, measurement of multiple genes can be advantageous as the measurement of multiple genes can increase the accuracy of diagnosis. This can be especially important where the diagnosis and monitoring is being performed by technicians in a clinical setting that may not be particularly versed in the techniques that are used to measure gene expression.

While increasing the number of genes in a panel will in general increase the accuracy, it will also increase the cost given that more reagents will be needed to perform the assay. However, careful selection of the gene sets in a panel for those genes that will provide the most information with the fewest total number can minimize the increase in cost while maximizing the increase in accuracy. In addition, two genes whose expression are correlated provide less information than two genes whose expression are not correlated, and if the degree of correlation is not taken into account in the analysis, then the results may appear more significant and therefore be misleading. Thus, there is a need for sets of genes whose expression is known to be correlated that will increase the accuracy of monitoring the functional status of transplants while requiring the smallest number of genes needed to attain such accuracy in the appropriate setting and limit misleading results. The present invention addresses these and other needs, and applies to functional status of transplants for which differential regulation of genes, or other nucleotide sequences, of peripheral blood can be demonstrated.

### SUMMARY

The present invention addresses these long felt needs by providing methods of monitoring the functional status of a transplant in a patient by detection of the expression level of a set of diagnostic genes. By monitoring the functional status of a transplant in a patient, the present invention provides more accuracy and can be more predictive than existing methods at predicting future graft dysfunction. The present invention further includes methods of generating such sets of diagnostic genes by selecting genes from multiple tables. In addition, the present invention provides compositions for use in practicing the foregoing methods and kits containing such compositions.

One aspect of the present disclosure is methods of diagnosing or monitoring the functional status of a transplant in a patient which includes detecting the expression levels of all genes of a diagnostic gene set in a patient wherein the diagnostic gene set includes at least one gene from each of at least two gene clusters chosen from the Cell-Surface Mediated Signaling Cluster, the Inflammation Cluster, the Steroid Responsive Gene Cluster, the Early Activation Cluster, the Heart Failure Cluster, the Hematopoiesis Cluster, the Megakaryocytes Cluster, the T/B Cell Regulation Cluster, the Transcription Control Cluster, the T Cell Cluster, the Inflammatory Cell Recruitment Cluster, the Transcription Factor Related Cluster, the Dendritic Cell Maturation Cluster, the Cell Activation Cluster, the Cytotoxic T Cell Cluster, and the Bone Marrow Stromal Cell Migration Cluster, and diagnosing or monitoring the functional status of a transplant in the patient based upon the expression levels of the genes in the diagnostic gene set. In certain embodiments, the gene clusters may be all genes whose expression is correlated with genes on the applicable table with a coefficient of correlation that is at least 0.25, 0.3 0.4, 0.5, 0.6, 0.7, 0.8, 0.9, 0.95, or 0.99.

In certain other embodiments, the gene clusters may be all genes listed on the corresponding table or all genes listed on the corresponding table together with applicable related diagnostic genes. In certain preferred embodiments, selected diagnostic genes or probes thereto may be selected from at least three different gene clusters, four different gene clusters, five different gene clusters, six different gene clusters, seven different gene clusters, eight different gene clusters, nine different gene clusters, ten different gene clusters, eleven different gene clusters, twelve different gene clusters, thirteen different gene clusters, fourteen different gene clusters, fifteen different gene clusters, or all sixteen gene clusters. In other embodiments, two or more diagnostic genes or probes thereto may be selected from a given cluster or three or more diagnostic genes or probes thereto may be selected from a given cluster.

In certain preferred embodiments, the transplant may be a cardiac transplant, a lung transplant, or a renal transplant. In various embodiments of the method, the expression levels of the diagnostic genes in the diagnostic gene set may be detected by the same method or by different methods. Certain preferred methods of detection include measuring the RNA level by hybridization to a labeled probe, hybridization to an array, and PCR amplification and detection, which may include use of oligonucleotides made from DNA, RNA, PNA, or mixtures thereof which may be prepared by synthetic methods or otherwise. In preferred embodiments, the RNA may be measured directly or may be converted to DNA first by any DNA polymerase that can use an RNA template. Certain other preferred methods of detection include measuring the protein level by measurement of the activity of the protein in an assay or by measurement using a labeled probe that interacts with the protein such as an antibody, binding partner or small molecule such as a substrate or cofactor.

In preferred embodiments the diagnosis or monitoring includes use of an algorithm that may be applied to the expression level. In certain preferred embodiments, the algorithm may be a cluster analysis algorithm, factor analysis algorithm, principal components and classification analysis algorithm, canonical analysis algorithm, classification trees analysis algorithm, multidimensional scaling analysis algorithm, discriminant function analysis algorithm, logistic regression algorithm, prediction analysis of microarrays (PAM) algorithm, voting algorithm (simple, smoothed and layered), TreeNet algorithm, random forests algorithm, and k-nearest neighbors algorithm.

In certain embodiments, the diagnosing or monitoring may be chosen from determining prognosis, determining risk of rejection or dysfunction, selecting therapeutic regimen, assessing ongoing therapeutic regimen, following progression of rejection or dysfunction. In certain variations, the therapeutic regimen may be one or more of various aspects such as selecting an immunosuppressant or other therapeutic agent, rejecting an immunosuppressant or other therapeutic agent, altering the dosage of an immunosuppressant or other therapeutic agent, selecting or rejecting additional diagnostic or monitoring assays or tests, identifying subsets of patients responsive to particular immunosuppressant or other therapeutic agent including positive response, no response or negative response such as adverse side effects. In certain embodiments, the methods of the present disclosure therefore include the additional step of altering the therapeutic regiment of a patient which for example may include selecting and/or applying additional diagnostic tests or assays, treating the patient with a new immunosuppressant or other therapeutic agent, altering the dosage of an immunosuppressant or other therapeutic agent.

Another aspect of the present disclosure includes methods of generating a probe set for diagnosing or monitoring the functional status of a transplant. Preferred embodiments of such methods involve generating a diagnostic gene set by selecting at least one gene from each of at least two gene clusters chosen from the Cell-Surface Mediated Signaling Cluster, the Inflammation Cluster, the Steroid Responsive Gene Cluster, the Early Activation Cluster, the Heart Failure Cluster, the Hematopoiesis Cluster, the Megakaryocytes Cluster, the T/B Cell Regulation Cluster, the Transcription Control Cluster, the T Cell Cluster, the Inflammatory Cell Recruitment Cluster, the Transcription Factor Related Cluster, the Dendritic Cell Maturation Cluster, the Cell Activation Cluster, the Cytotoxic T Cell Cluster, and the Bone Marrow Stromal Cell Migration Cluster, and generating a probe set by creating at least one probe that specifically detects the expression level for each gene in the diagnostic gene set. In certain embodiments, the gene clusters may be all genes whose expression is correlated with genes on the applicable table with a coefficient of correlation that is at least 0.25, 0.3 0.4, 0.5, 0.6, 0.7, 0.8, 0.9, 0.95, or 0.99.

In certain other embodiments, the gene clusters may be all genes listed on the corresponding table or all genes listed on the corresponding table together with applicable related diagnostic genes. In certain preferred embodiments, selected diagnostic genes or probes thereto may be selected from at least three different gene clusters, four different gene clusters, five different gene clusters, six different gene clusters, seven different gene clusters, eight different gene clusters, nine different gene clusters, ten different gene clusters, eleven different gene clusters, twelve different gene clusters, thirteen different gene clusters, fourteen different gene clusters, fifteen different gene clusters, or all sixteen gene clusters. In other embodiments, two or more diagnostic genes or probes thereto may be selected from a given cluster or three or more diagnostic genes or probes thereto may be selected from a given cluster.

In yet other embodiments, the transplant may be a cardiac transplant, a lung transplant or a renal transplant. In various embodiments of the method, the probe for measuring the expression levels of the diagnostic genes in the diagnostic gene set may be different types of probes or the same type of probe. Certain preferred probes include oligonucleotides that may be used to hybridize to the RNA or cDNA of a diagnostic gene direct detection in solution or affixed to a solid support such as an array or membrane, or use as a primer for amplification and later detection. Preferred examples of such oligonucleotide include oligonucleotides made from DNA, RNA, PNA, or mixtures thereof which may be prepared by synthetic methods or otherwise. Certain other preferred probes include antibodies or other proteins such as binding partners that bind specifically to the gene product of the diagnostic genes and small molecules such as labeled substrate or cofactors for binding or activity assays.

Another aspect of the present disclosure includes the probe sets that are generated by the aforementioned methods or that otherwise meet the above descriptions. In certain embodiments the probe sets may be included in kit that may have instructions for the use, software embodying any algorithm to be used in diagnosis or monitoring, buffers and/or enzymes used in preparation of RNA, cDNA or protein samples as appropriate and buffers and/or enzymes used in detection of such RNA, cDNA or protein samples as appropriate.

In certain aspects of the present disclosure, diagnostic gene sets may include two or more genes selected from at least one gene cluster chosen from the Cell-Surface Mediated Signaling Cluster, the Inflammation Cluster, the Steroid Responsive Gene Cluster, the Early Activation Cluster, the Heart Failure Cluster, the Hematopoiesis Cluster, the Megakaryocytes Cluster, the T/B Cell Regulation Cluster, the Transcription Control Cluster, the T Cell Cluster, the Inflammatory Cell Recruitment Cluster, the Transcription Factor Related Cluster, the Dendritic Cell Maturation Cluster, the Cell Activation Cluster, the Cytotoxic T Cell Cluster, and the Bone Marrow Stromal Cell Migration Cluster. In certain preferred embodiments, such diagnostic gene sets may include three or more, four or more, five or more, six or more, or eight or more genes selected from at least one gene clusters. These diagnostic gene sets may be used in all of the various aspects and embodiments listed above.

Another class of embodiments of the present disclosure is the use of the sixteen gene subclusters rather than the sixteen gene clusters. The gene subclusters may be identified by reference to Tables 1-16 in column 2, which preferably is limited to either the genes on Tables 1-16 which are listed in parenthesis or the genes on Tables 1-16 which are not listed in parenthesis. By way of example, the three alternate gene subclusters for gene subcluster 2 are as follows: (1-all) CLC, MME, MMP9, CD24, A_{—}32_P100109, LIN7A, SC100A12, SCL22A16, CA4, CEBPE, ORM1, and ACSL1; (2—listed in parenthesis) CD24, A_{—}32_P100109, LIN7A, SC100A12, SCL22A16, CA4, CEBPE, ORM1, and ACSL1; and (3—not listed in parenthesis) CLC, MME, MMP9. The gene subclusters may be used in place of the gene clusters as alternate embodiments throughout the disclosure herein.

In another class of embodiments, one of the diagnostic genes may be selected from a table, cluster or subcluster that was identified by microarray only as is designated by “M” in column 3 of Tables 1-16. Such additional diagnostic gene may used in conjunction with any of the methods and compositions disclosed herein.

Yet another class of embodiments is use of the sixteen clusters (or subclusters) where one diagnostic gene from a cluster (or subcluster) is specified and then addition diagnostic genes are selected from the remaining clusters. By way of example, preferred diagnostic genes that may be “fixed” while selecting from the other clusters or subclusters are ITGA4, MMP9, IL18, IL1R2, FLT3, CPM, EPB42, WDR40A, HBA1, ALAS2, ITGA2B, MPL, INPP5A, TNFSF4, SELP, IL7R, TNFRSF7, FLT3LG, CD28, PDCD1, CD160, CD8B1, CD8A, GZMB, PRF1, GNLY, LCK, CXCR3, GATA3, ITGB7, KPNA6, and NOTCH1. Thus an example of such a set would be ITGA4, the Inflammation Cluster, the Steroid Responsive Gene Cluster, the Early Activation Cluster, the Heart Failure Cluster, the Hematopoiesis Cluster, the Megakaryocytes Cluster, the T/B Cell Regulation Cluster, the Transcription Control Cluster, the T Cell Cluster, the Inflammatory Cell Recruitment Cluster, the Transcription Factor Related Cluster, the Dendritic Cell Maturation Cluster, the Cell Activation Cluster, the Cytotoxic r Cell Cluster, and the Bone Marrow Stromal Cell Migration Cluster.

Additional preferred aspects and embodiments of the present disclosure may be found in the detailed description of the preferred embodiments below.

### BRIEF DESCRIPTION OF THE DRAWINGS

FIG. 1: Analysis of Future Graft Dysfunction. (A) Biopsy grade 0 samples (n=61): future graft dysfunction (PCW>20 mm Hg within 45 days) in the ≧4 month post-transplant cohort predicted by incidence of high (≧18.5) and low (<18.5) algorithm scores. The p-value shown is from the Fisher Exact test. The average PCW values for the two groups at the time of the test sample were the same (10.6 and 9.7 for those with and without future graft dysfunction, respectively). (B) Biopsy grade 0 and ≧3A samples (n=91): future graft dysfunction (PCW>20 mm Hg within 45 days) in the ≦4 month post-transplant cohort predicted by algorithm score versus biopsy grade.

FIG. 2: Longitudinal case studies. The discriminant algorithm score from Example 4 ranging from 0 to 40 is plotted on the y-axis for each post-transplant visit. The associated ISHLT biopsy grade is associated with each visit. (A) Quiescent patient. This patient had 9 endomyocardial biopsies during the 1st 800 days post transplant, all of which were below a score of 28. T]he patient had a benign clinical course. (B) Rejector patient. The patient had 7 ISHLT Grade 0 or 1A biopsies in the 1st 300 days associated with low algorithm scores. An algorithm score above 30 which is associated with a Grade 1A biopsy precedes a Grade 3A rejection which is treated with bolus corticosteroids (arrow). The patient subsequently died of multi-organ system failure and sepsis.

FIG. 3: Prediction of Acute Cellular Rejection Study. This figure shows the overall design of the study performed in Example 5.

### DESCRIPTION OF THE TABLES

The following tables are included in this specification:
Table 1: This table lists genes in the Cell-Surface Mediated Signaling Cluster.
Table 2: This table lists genes in the Inflammation Cluster.
Table 3: This table lists genes in the Steroid Responsive Gene Cluster.
Table 4: This table lists genes in the Early Activation Cluster.
Table 5: This table lists genes in the Heart Failure Cluster.
Table 6: This table lists genes in the Hematopoiesis Cluster.
Table 7: This table lists genes in the Megakaryocytes Cluster.
Table 8: This table lists genes in the T/B Cell Regulation Cluster.
Table 9: This table lists genes in the Transcription Control Cluster.
Table 10: This table lists genes in the T Cell Cluster.
Table 11: This table lists genes in the Inflammatory Cell Recruitment Cluster.
Table 12: This table lists genes in the Transcription Factor Related Cluster.
Table 13: This table lists representative genes in the Dendritic Cell Maturation Cluster.
Table 14: This table lists genes in the Cell Activation Cluster.
Table 15: This table lists genes in the Cytotoxic T Cell Cluster.
Table 16: This table lists genes in the Bone Marrow Stromal Cell Migration Cluster.

Tables 1 through 16 lists genes in each of the sixteen clusters defined herein. Each table includes the minimum pair-wise correlation between the expression of the genes in the cluster in column one. Each table includes the subcluster originally determined with RT-PCR expression level data only. Each table also includes the source of the gene (P—RT-PCR; M—Microarray; and B—Both). Each table includes the gene symbol for such gene as used in the Entrez Gene database in column three. One skilled in the art can use the gene symbols to obtain genomic and transcript sequence information, domain structure, and a bibliography of publications relating to the gene from the Entrez Gene database. The Entrez Gene database has been implemented at the National Center for Biotechnology Information (NCBI) to organize information about genes, serving as a major node in the nexus of genomic map, sequence, expression, protein structure, function, and homology data. Each Entrez Gene record is assigned a unique identifier, the GenelD that can be tracked through revision cycles. The Entrez Gene database is publicly accessible at ncbi.nlm.nih.gov. The current web page may be accessed at the site at “entrez/query.fcgi?db=gene&cmd=search&term=”. Each table includes the annotation for such gene obtained from the Entrez Gene database in column four. For genes that do not list the Entrez Gene symbol, the Agilent probe number is provided with the annotation which one of skill in the art may readily use to identify the particular gene.
Table 17: This table lists cutoffs for the simple voting algorithm provided in Example 1.
Table 18: This table lists coefficients for the alternative voting algorithm provide in Example 1.
Table 19: This table lists coefficients for the logistic regression algorithm provided in Example 2.
Table 20: This table lists coefficients for the first linear algorithm provided in Example 3.
Table 21: This table lists coefficients for the second linear algorithm provided in Example 3.
Table 22: This table lists coefficients for the third linear algorithm provided in Example 3.
Table 23: Clinical Characteristics of Patient Populations. This table provides a comparison of clinical parameters of patients and samples used in the microarray, training and validation studies in Example 4. Abbreviations: CARGO (Cardiac Allograft Rejection Gene expression Observation study) and UNOS (United Network for Organ Sharing).
Table 24: Discriminant Algorithm Performance. This table provides the discriminant algorithm performance relative to a biopsy standard with single a priori estimated threshold and time-dependent (≦4 month and >4 month) thresholds are shown for Example 4. Performance estimates in later periods post-transplant (>6 months, >12 months) are also included. Performance is given as % agreement with biopsy Grade 3A or Grade 0 defined by centralized reading. Agreement rates are reported for the PCR training study using bootstrap estimates, for the validation set and for the sets of samples from patients unique to the validation study (Validation unique).

### DETAILED DESCRIPTION OF THE PREFERRED EMBODIMENTS

The present disclosure provides sets of genes organized into clusters that are useful in the detection and monitoring of the functional status of transplants in patients. Genes may be selected from the different clusters of diagnostic genes to create a diagnostic gene set. The diagnostic gene set may be used to monitor the functional status of a transplant in a patient by measuring the expression level of the genes in the diagnostic gene set over time. Monitoring the functional status of a transplant in a patient is particularly useful for detecting rejection and other graft dysfunction in that patient by measuring the expression levels of the diagnostic gene set in a sample obtained from an individual. Methods of using the diagnostic gene sets including detection methods and analysis methods are also described herein. The expression pattern of the diagnostic gene set may be further analyzed by application of algorithms to monitor the status of the individual. By way of example, such analysis can determine whether the individual is rejecting a transplanted organ, tissue or cell sample, how the individual is responding to an immunosuppressant, and how the individual is responding to therapy to treat rejection of a transplanted organ, tissue or cell sample. More importantly, the present disclosure provides a more sensitive measure of the functional status of a transplant in a patient and can therefore provide a predictive indication of the patient's near term status. Such predictive indications can be used as a basis to adjust the immunosuppressant regimen of a patient to prevent rejection as well as minimize the bad effects of over immunosuppression by allowing fine tuning of the immunosuppressant regimen. As is demonstrated in the Examples, the present disclosure provides methods that are more accurate at predicting heart transplant dysfunction than the current method of analyzing biopsies. The present invention further provides preferred methods to analyze the expression patterns of the diagnostic gene sets.

The diagnostic gene clusters of the present disclosure were determined based upon the correlation between the expression of the diagnostic genes. Each cluster represents a group of diagnostic genes whose expression is correlated or is likely to be correlated. Therefore, selecting multiple genes from the same cluster may increase the precision of the measurement without necessarily improving the accuracy of the prediction. Similarly, selecting multiple genes from different clusters will increase the accuracy of the prediction without necessarily increasing the precision of the measurement. Using these two general principles, one of skill in the art can fine tune the diagnostic gene set based upon the need for accuracy vs. precision. In addition, selecting genes from multiple pathways may decrease false positives where one cluster may be activated but in response to a stimulus other than transplant rejection.

### DEFINITIONS

Unless defined otherwise, all scientific and technical terms are understood to have the same meaning as commonly used in the art to which they pertain. The following terms are defined below.

In the context of the disclosure, the term “gene expression system” refers to any system, device or means to detect gene expression and includes diagnostic agents, candidate libraries, oligonucleotide sets or probe sets.

The term “monitoring” is used herein to describe the use of gene sets to provide useful information about an individual or an individual's health or disease status. “Monitoring” can include, determination of prognosis, risk-stratification, selection of drug therapy, assessment of ongoing drug therapy, prediction of outcomes, determining response to therapy, diagnosis of a disease or disease complication, following progression of a disease or providing any information relating to a patients health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example a cascade of tests from a non-invasive blood test to a more invasive option such as biopsy, or testing to assess side effects of drugs used to treat another indication.

The “functional status of a transplant” covers all biological and physiological aspects of a transplant including the immune status. The immune status includes the degree and nature of immune related complications such as cellular rejection (acute), humoral rejection, and chronic rejection (vasculopathy, chronic allograft nephropathy, bronchiolitis obliterans syndrome). The functional status includes measures of all parameters of the transplanted organ, tissue or cells processes as well as all dysfunction associated with the transplant

Immunosuppressants for which the present disclosure may diagnose treatment with or exclude from treatment include cyclosporin A, everolimus, tacrolimus (FK506), rapamycin (sirolimus), azathioprine, mycophenolate mofetil (MMF), methotrexate, campath-1H, an anti CD52 antibody, OKT3 (anti CD3 antibody), OKT4, anti-TAC, prednisone or other corticosteroids, alpha lymphocyte antibodies, thymoglobulin, brequinar sodium, leflunomide, CTLA-4 Ig, an anti-CD25 antibody, an anti-IL2R antibody, basiliximab, daclizumab, mizoribine, FK 778, ISAtx-247, hu5C8, etanercept, adalimumab, infliximab, LFA3Ig, natalizumab, cyclophosphamide, deoxyspergualin, tresperimus, UO126, B7RP-1-fc, and NOX-100.

A “gene” as used herein refers to any RNA that is transcribed from DNA in an organism including, without limitation, humans. Thus, a gene includes by way of example, but not limitation, mRNA, tRNA, rRNA, hnRNA, and mRNA processing intermediates.

A “diagnostic gene” is a gene whose expression correlates to the functional status of an organ in a transplant patient. The expression of a diagnostic gene may be detected by a diagnostic oligonucleotide or other method directed to detecting RNA or protein produced therefrom and such expression may be used to monitor transplant rejection or inflammation based disorders in a patient.

A “diagnostic gene set” is a-set of diagnostic genes whose expression may be detected by a diagnostic oligonucleotide or other method directed to detecting RNA or protein produced therefrom and such expression may be used to monitor functional status of a transplant including transplant rejection or used to monitor inflammation based disorders in a patient. A diagnostic gene set may be generated by selecting at least two diagnostic genes where each gene is selected from a different cluster (or table as described herein). In a preferred embodiment, the diagnostic gene is generated by selecting at least three diagnostic genes where each gene is selected from a different cluster. In a more preferred embodiment, the diagnostic gene is generated by selecting at least four diagnostic genes where each gene is selected from a different cluster. In a yet more preferred embodiment, the diagnostic gene is generated by selecting at least five diagnostic genes where each gene is selected from a different cluster. In an even more preferred embodiment, the diagnostic gene is generated by selecting at least six diagnostic genes where each gene is selected from a different cluster. In certain variations, additional genes may be selected from a cluster from which a gene has already been selected. It is understood that the use of “diagnostic” in the terms diagnostic gene and diagnostic gene set is not intended to limit the use to diagnosis, but rather the diagnostic genes and diagnostic genes sets may be used for the full range of activities that relate to gene expression monitoring.

The diagnostic genes described herein are divided into sixteen clusters or gene clusters. For convenience, the sixteen clusters have been organized into sixteen tables. The diagnostic genes were grouped into these sixteen clusters based upon the correlation in the change in expression of the diagnostic genes in response to changes in the immune status of individuals with transplants. The genes in the present clusters were identified by selection from microarray experiments as well as QPCR on clinical samples. Gene selection from microarrays was accomplished by Statistical Analysis of Microarrays (SAM), hierarchical clustering by Cluster3 and data visualization by Java TreeView and non-parametric analysis (Fischer exact). QPCR data analysis was accomplished by with Student's t-test, median ratios, hierarchical clustering by Cluster3 and data visualization by JavaTreeView.

As used herein the term “gene cluster” or “cluster” refers to a group of genes related by expression pattern. A cluster of genes is a group of genes with similar regulation across different conditions, such as graft non-rejection versus graft rejection. In a preferred embodiment, the expression of the diagnostic genes in a cluster or gene cluster has a correlation coefficient of at least 0.3 with regard to the other genes in the cluster. In a more preferred embodiment, the correlation coefficient is at least 0.3 0.4, 0.5, 0.6, 0.7, 0.8, 0.9, 0.95, or 0.99.

A “probe set” as used herein refers to a group of nucleic acids that may be used to detect two or more genes. Probes in a probe set may be labeled with one or more fluorescent, radioactive or other detectable moieties (including enzymes). Probes may be any size so long as the probe is sufficiently large to selectively detect the desired gene. A probe set may be in solution, as would be typical for multiplex PCR, or a probe set may be adhered to a solid surface as in an array or microarray. In addition, probes may contain rare or unnatural nucleic acids such as inosine.

The diagnostic genes listed in tables 1-16 were assigned to the clusters based upon the correlation between the expression of the genes (i.e., genes whose expression is correlated were included in the same cluster). The same methods used to assign the present diagnostic genes to the clusters may be used to add additional diagnostic genes to the clusters. Additional diagnostic genes may be included in a cluster based upon a correlation between expression of additional diagnostic genes and the expression of the genes included on one of the sixteen tables. Any suitable statistical analysis method for calculation of correlation may be used. Examples of suitable methods Pearson correlation (which calculates the coefficient between any two series of gene expression profiles), Spearman rank correlation (which calculates the correlation between the rank and magnitude of the data values), Kendall's tau (which calculates the correlation between the relative ordering of ranks), Euclidean distance (which calculates the correlation based on the magnitude of changes in gene expression levels), City-block distance (also known as Manhattan distance, which calculates the sum of distances along each dimension). In a preferred embodiment, the additional diagnostic genes in a cluster have a correlation coefficient of at least 0.25 with regard to the genes in the table corresponding to the cluster. In a more preferred embodiment, the correlation coefficient is at least 0.3 0.4, 0.5, 0.6, 0.7, 0.8, 0.9, 0.95, or 0.99.

“Related diagnostic genes” of a table or cluster are those genes whose expression are correlated to the expression of those genes in a given table or cluster that interact directly with one or more of the diagnostic genes on the table or cluster. Such interacting genes are very readily identifiable by one of skill in the art owing to the direct interaction. Examples of such direct interaction include hemoglobin HBA and HBB proteins. The inventors initially identified hemoglobin HBA protein as belonging to the Hematopoiesis Cluster and recognized that hemoglobin HBB protein would therefore also belong in the Hematopoiesis Cluster. As expected, the expression of HBB proteins did correlate and was added.

Functional status monitoring for transplants is a complex process involving many immune cell types as well as many pathways within cells. The multiple gene clusters described herein allow simultaneous monitoring and assessment of these multiple cell types and pathway. As one would expect, certain clusters with correlated expression have a clear underlying biological basis for their correlation. By way of example, those genes expressed in specific cell type such as B-cells or T-cells are in the same cluster. Another example of a clear biological relationship would be a set of enzymes in an enzymatic pathway such as enzymes in an enzymatic pathway for synthesizing a cofactor of a diagnostic gene such as heme biosynthetic enzymes in the Hematopoiesis Cluster. Given the biological relationship of the genes within such clusters, one of skill in the art would have no difficulty in identifying additional diagnostic genes that would fall within such clusters and demonstrating that the expression of such additional diagnostic genes correlates with the diagnostic genes in the relevant cluster. The underlying biological relationship of each cluster is described for each group below.

The “Cell-Surface Mediated Signaling Cluster” includes all diagnostic genes with correlated expression that are associated with cell to cell signaling and cell adhesion. Representative diagnostic genes in the Cell-Surface Mediated Signaling Cluster are listed in Table 1. One of skill in the art will recognize that molecules in this cluster are involved in cellular processes which involves signal transduction, and therefore in addition to those genes listed in Table 1, other genes are involved in signal transduction will have correlated expression and therefore belong in this cluster.

The “Inflammation Cluster” includes all diagnostic genes with correlated expression that are associated with catalytic activity related to inflammation. Representative diagnostic genes in the Inflammation Cluster are listed in Table 2. One of skill in the art will recognize that molecules in this cluster are involved in hydrolase activity specifically metalloendopeptidase activity, and therefore in addition to those genes listed in Table 2, other genes are have such hydrolase activity will have correlated expression and therefore belong in this cluster.

The “Steroid Responsive Gene Cluster” includes all diagnostic genes with correlated expression that are associated with inflammatory response associated with steroids. Representative diagnostic genes in the Steroid Responsive Gene Cluster are listed in Table 3. One of skill in the art will recognize that molecules in this cluster are involved in response to steroid in relation to inflammation, and therefore in addition to those genes listed in Table 3, other genes are involved in such response will have correlated expression and therefore belong in this cluster. Genes in this cluster are often expressed by neutrophils and monocytes.

The “Early Activation Cluster” includes all diagnostic genes with correlated expression that are associated with the defense response. Representative diagnostic genes in the Early Activation Cluster are listed in Table 4. One of skill in the art will recognize that molecules in this cluster are involved in migration of cells from the bone marrow during early activation of the immune system, and therefore in addition to those genes listed in Table 4, other genes are involved in such migration will have correlated expression and therefore belong in this cluster.

The “Heart Failure Cluster” includes all diagnostic genes with correlated expression that are associated with heart failure. Representative diagnostic genes in the Heart Failure Cluster are listed in Table 5. One of skilled in the art will recognize that molecules in this cluster are associated with heart failure, and therefore in addition to those genes listed in Table 5, other genes are involved in heart failure will have correlated expression and therefore belong in this cluster.

The “Hematopoiesis Cluster” includes all diagnostic genes with correlated expression that are associated with the erythroid lineage leading to red blood cells. Representative diagnostic genes in the Hematopoiesis Cluster are listed in Table 6. One of skill in the art will recognize that molecules in the heme biosynthetic pathway, such as ALAS2, will have correlated expression with the genes on Table 6 and therefore belong in this cluster as well as the globins themselves. In addition, molecules involved in transport of heme precursors will have correlated expression with the genes in Table 6 as well. It is worth noting that the Hematopoiesis Cluster is particularly effective at detecting the shift to production of immature red blood cells such as reticulocytes, which are useful for monitoring various immune related disorders. The up-regulation of expression of genes in this cluster associated with transplant rejection may be responsive to hypoxia and/or graft dysfunction.

The “Megakaryocytes Cluster” includes all diagnostic genes with correlated expression that are specifically expressed in Megakaryocytes or platelets. Representative diagnostic genes in the Megakaryocytes Cluster are listed in Table 7.

The “T/B Cell Regulation Cluster” includes all diagnostic genes with correlated expression that are associated with immune response. Representative diagnostic genes in the T/B Cell Regulation Cluster are listed in Table 8. One of skill in the art will recognize that molecules in this cluster are involved in B and T cell differentiation and in T cell costimulation as exemplified by CD28.

The “Transcription Control Cluster” includes all diagnostic genes with correlated expression that are associated with nuclear functions. Representative diagnostic genes in the Transcription Control Cluster are listed in Table 9. One of skill in the art will recognize that molecules in this cluster are involved in nuclear transport, telomere maintenance and response to DNA damage, and therefore in addition to those genes listed in Table 9, other genes involved in such nuclear functions will have correlated expression and therefore belong in this cluster.

The “T Cell Cluster” includes all diagnostic genes with correlated expression that are associated with T cells. Representative diagnostic genes in the T Cell Cluster are listed in Table 10. One of skill in the art will recognize that molecules in this cluster are involved in activated T cell proliferation, especially in CD8⁺ T cell proliferation, and therefore in addition to those genes listed in Table 10, other genes involved in T cell proliferation will have correlated expression and therefore belong in this cluster.

The “Inflammatory Cell Recruitment Cluster” includes all diagnostic genes with correlated expression that are associated with intracellular signaling. Representative diagnostic genes in the Inflammatory Cell Recruitment Cluster are listed in Table 11. One of skilled in the art will recognize that molecules in this cluster are involved in recruitment of immune cells, and therefore in addition to those genes listed in Table 11, other genes involved in such recruitment will have correlated expression and therefore belong in this cluster.

The “Transcription Factor Related Cluster” includes all diagnostic genes with correlated expression that are associated with transcription factor activity. Representative diagnostic genes in the Transcription Factor Related Cluster are listed in Table 12.

The “Dendritic Cell Maturation Cluster” includes all diagnostic genes with correlated expression that are associated with cell differentiation and development of dendritic cells and NKT cells as exemplified by CD1D expression. Representative diagnostic genes in the Dendritic Cell Maturation Cluster are listed in Table 13, other genes associated with cell differentiation of dendritic cells and NKT cells will have correlated expression and therefore belong in this cluster.

The “Cell Activation Cluster” includes all diagnostic genes with correlated expression that are associated with cell trafficking of various types of leukocytes. Representative diagnostic genes in the Cell Activation Cluster are listed in Table 14, other genes associated with cell trafficking of leukocytes will have correlated expression and therefore belong in this cluster.

The “Cytotoxic T Cell Cluster” includes all diagnostic genes with correlated expression that are associated with cytolysis. Representative diagnostic genes in the Cytotoxic T Cell Cluster are listed in Table 15. One of skill in the art will recognize that molecules in this cluster are involved in T cells or NK cells involved in cell killing, and therefore in addition to those genes listed in Table 15, other genes specifically expressed in Cytotoxic T cells will have correlated expression and therefore belong in this cluster.

The “Bone Marrow Stromal Cell Migration Cluster” includes all diagnostic genes with correlated expression that are associated with pro-inflammatory activity. Representative diagnostic genes in the Bone Marrow Stromal Cell Migration Cluster are listed in Table 16, other genes associated with pro-inflammatory activity will have correlated expression and therefore belong in this cluster.

In the context of the present disclosure, a “patient” may be an individual who has received any form of transplanted material that may be recognized by the individual's immune system as foreign or may otherwise stimulate an inflammation response. The transplanted material may include organs, tissues and cells from another individual or from an animal of a different species. Such transplanted material may also include the individuals own tissues or cells after a modification that renders such material as foreign to the individual's immune system including, by way of example, transgenic manipulation. Such transplant material may also include artificial implants such as mechanical replacement organs. By way of example, transplant rejection that may be monitored by the methods described herein include heart transplant rejection, kidney transplant rejection, liver transplant rejection, pancreas transplant rejection, pancreatic islet transplant rejection, lung transplant rejection, bone marrow transplant rejection, stem cell transplant rejection, xenotransplant rejection, and mechanical organ replacement rejection.

A “patient sample” includes any suitable sample taken from a recipient of a transplant from which expression of a diagnostic gene set may be measured. Such samples may be obtained by any means available to one of ordinary skill in the art. Preferred samples are those that will include leukocytes due to the relationship between leukocytes and transplant rejection. By way of example, circulating leukocytes from whole blood from the peripheral vasculature may be used as such sampling is generally the simplest, least invasive, and lowest cost alternative. However, no significant distinction exists, in fact, between leukocytes sampled from the peripheral vasculature, and those obtained, e.g., from a central line, from a central artery, or indeed from a cardiac catheter, or during a surgical procedure which accesses the central vasculature. In addition, other body fluids and tissues that are, at least in part, composed of leukocytes are also preferred leukocyte samples. For example, fluid samples obtained from the lung during bronchoscopy and bronchoalveolar lavage may be rich in leukocytes, and amenable to expression profiling in the context of the disclosure, e.g., for the diagnosis, prognosis, or monitoring of lung transplant rejection, inflammatory lung diseases or infectious lung disease. Fluid samples from other tissues, e.g., obtained by endoscopy of the colon, sinuses, esophagus, stomach, small bowel, pancreatic duct, biliary tree, bladder, ureter, vagina, cervix or uterus, etc., are also suitable. Samples may also be obtained other sources which may or may not contain leukocytes, e.g., from urine, bile, or solid organ or joint biopsies.

Generation of a Diagnostic Gene Set

Selection of a set of diagnostic genes from the tables together with related diagnostic genes or from the clusters can be done by one of skill in the art with little difficulty. Since each table or cluster represents a group of coordinately regulated genes, selection of one or more diagnostic genes from each of three or more tables or clusters would generate a set of diagnostic genes that would be useful for monitoring the functional status of a transplant in a patient. A preferred method of selecting genes is to perform a multivariate analysis of a larger family of diagnostic genes to identify those that provide the greatest degree of information such as by identifying genes whose expression yields the greatest separation between transplant patients that are and are not rejecting their transplant. Those that provide the greatest degree of information may be used in the final diagnostic gene set. Final selection would be based upon principles such as selecting a sufficient number of genes from different tables or clusters to provide non-redundant information and pairing genes from the same table or cluster to increase the accuracy of smaller differences that are still statistically relevant. One of skill in the art would have no difficulty in determining the appropriate classes to measure separation based upon what is to be monitored in the patients, e.g., in measuring transplant rejection one could measure separation between patients with quiescent immune systems (non-rejecters) and patients with immune activation (rejecters). Further, any useful metric of separation may be used in such analysis. By way of example, significant differences in mean, such as t-test, or median ratios or the differences in the mean of two classes have to exceed the natural variation (the separation within the class). Separation can also be determined by dividing the difference of the mean or median by the sum of the standard deviation of each class. Multiple metrics can even be used. For example, a simple test could be used to remove the less informative diagnostic gene and then a more complicated test could be used to identify which among the more informative diagnostic genes are the most informative. An even more preferred method is to combine selection of the diagnostic gene set with the determination of an algorithm to be used in monitoring a patient.

Preparation of a Patient Sample for Detection of Expression Levels

A patient sample may be processed in preparation of detecting the expression levels the diagnostic gene sets by any technique available to one of skill in the art. Selection of the further processing will depend upon the method(s) of detection to be used in detection of expression. Given that expression levels can be evaluated at the level of DNA, or RNA or protein products, the further processing may involve purification and, in the case of RNA amplification of the desired product. For example, a variety of techniques are available for the isolation of RNA from whole blood or other patient samples. Any technique that allows isolation of mRNA from cells (in the presence or absence of rRNA and tRNA) can be utilized. In brief, one method that allows reliable isolation of total RNA suitable for subsequent gene expression analysis is described as follows. Peripheral blood (either venous or arterial) is drawn from a subject, into one or more sterile, endotoxin free, tubes containing an anticoagulant (e.g., EDTA, citrate, heparin, etc.). Typically, the sample is divided into at least two portions. One portion, e.g., of 5-8 ml of whole blood is frozen and stored for future analysis, e.g., of DNA or protein. A second portion, e.g., of approximately 8 ml whole blood is processed for isolation of total RNA by any of a variety of techniques as described in, e.g., Sambook, Ausubel, below, as well as U.S. Pat. Nos. 5,728,822 and 4,843,155 and use of the RNeasy Mini Kit™ (Cat. No. 74106, Qiagen) and RNase-Free DNase Set™ (Cat. No. 79254, Qiagen) following the recommended procedures therein.

Amplification may be achieved using standard techniques such as PCR, linear amplification may be performed, as described in U.S. Pat. No. 6,132,997, rolling circle amplification, etc. Further, amplification and detection may be combined as in TaqMan™ real-time PCR detection such as with the TaqMan Assay™ on the ABI 7900HT™ following the recommended protocols. Variability may be controlled for by adding a positive control amplification in one, two, three, four or more of the wells. Such positive control may be from any organism such a bacterial gene, a plant gene, or an animal gene.

Measuring Expression of a Diagnostic Gene Set

The expression levels of the diagnostic gene set may be measured by any means available to one of skill in the art, including without limitation RNA profiling such as Northern analysis, PCR, RT-PCR, TaqMan analysis, FRET detection, hybridization to an oligonucleotide array, hybridization to a cDNA array, hybridization to a polynucleotide array, hybridization to a liquid microarray, hybridization to a microelectric array, molecular beacons, etc. as well as immunoassay, fluorescent activated cell sorting, protein assay, enzyme assay, peripheral blood cytology assay, MRI imaging, bone marrow aspiration, and/or nuclear imaging.

In addition, expression may be measured at the level of protein products of the diagnostic genes of the diagnostic gene set. For example, protein expression in a sample can be evaluated by one or more method selected from among: Western analysis, two-dimensional gel analysis, chromatographic separation, mass spectrometric detection, protein-fusion reporter constructs, colorimetric assays, binding to a protein array and characterization of polysomal mRNA. Methods for producing and evaluating antibodies are widespread in the art, see, e.g., Coligan, supra; and Harlow and Lane (1989) Antibodies: A Laboratory Manual, Cold Spring Harbor Press, NY (“Harlow and Lane”). Additional details regarding a variety of immunological and immunoassay procedures adaptable to the present methods and compositions by selection of antibody reagents specific for the products of candidate nucleotide sequences can be found in, e.g., Stites and Terr (eds.) (1991) Basic and Clinical Immunology, 7th ed., and Paul, supra. Another approach uses systems for performing desorption spectrometry. Alternatively, affinity reagents (e.g., antibodies, small molecules, etc.) are available that recognize epitopes of the protein product. Affinity assays are used in protein array assays, e.g. to detect the presence or absence of particular proteins. Alternatively, affinity reagents are used to detect expression using the methods described above. In the case of a protein that is expressed on the cell surface of leukocytes, labeled affinity reagents are bound to populations of leukocytes, and leukocytes expressing the protein are identified and counted using fluorescent activated cell sorting (FACS).

One of skill in the art would select the appropriate method of measurement based upon such factors as type of transplant rejection, ease of measurement of each particular diagnostic gene, need for accuracy of measurement of each particular gene, etc. When measuring the expression at the protein level, selection of the technique will be dictated by the nature of the protein, e.g., activity assays are useful for enzymes, fluorescent activated cell sorting is useful for membrane bound and membrane associated proteins. In certain embodiments, different techniques may be used to measure each diagnostic gene in a set. In other embodiments, the same technique may be used to measure expression of all the genes in the diagnostic gene set.

The disclosure also provides diagnostic probe sets used for detecting the expression levels of the diagnostic genes of the diagnostic gene set. It is understood that a probe includes any reagent capable of specifically identifying a nucleotide sequence of a given diagnostic gene in a diagnostic gene set, including but not limited to DNA, RNA, cDNA, synthetic oligonucleotide, partial or full-length nucleic acid sequences. In addition, the probe may identify the protein product of a diagnostic gene, including, for example, antibodies and other affinity reagents. Further, an individual probe can correspond to one gene and multiple probes can correspond to one gene. Such probes may be used in any combination in detecting the expression levels of a diagnostic gene set. By way of example, a diagnostic gene set that has four diagnostic genes may have a diagnostic probe that detects the first diagnostic gene, three diagnostic probes that detect the second diagnostic gene, two diagnostic probe that detects the third gene and a seventh diagnostic probe that detects the fourth gene. The seven diagnostic probes in such example constitute the diagnostic probe set.

In some embodiments, a diagnostic probe set is immobilized on an array. The array is optionally includes one or more of: a chip array, a plate array, a bead array, a pin array, a membrane array, a solid surface array, a liquid array, an oligonucleotide array, a polynucleotide array or a cDNA array, a microtiter plate, a pin array, a bead array, a membrane or a chip.

Where the probe set detects expression levels by hybridization to nucleic acids, hybridization conditions may be highly stringent or less highly stringent, depending upon the required specificity. By way of example, where the probe set is hybridized to RNA samples including RNA samples that have been amplified and/or converted to DNA by reverse transcriptase, highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos).

Algorithms

The results of measuring the expression of the diagnostic gene set may be analyzed by any means available to one of skill in the art. Typically an algorithm will be applied to the data. Such algorithms may be as simple, for example, as a doctor or other medical professional comparing expression levels of a diagnostic gene set measured in a sample to a reference card with ranges of expression to determine whether a threshold number of diagnostic genes fall in a certain prescribed range of expression. In addition, simple pair-wise comparison algorithms may be used; however, multivariate analysis is preferred with larger diagnostic gene sets. Many algorithms suitable for multivariate analysis are known in the art such as Cluster Analysis, Factor Analysis, Principal Components & Classification Analysis, Canonical Analysis, Classification Trees Analysis, Multidimensional Scaling Analysis, Discriminant Function Analysis, (StatSoft Inc.), logistic regression (SAS Institute Inc.), Prediction Analysis of Microarrays (PAM), voting (simple, smoothed and layered), TreeNet (Salford Systems), Random Forests, and k-nearest neighbors.

A preferred example of such an algorithm is where a set of genes shown to discriminate between rejection and quiescence in the training phase of the study are allowed to ‘vote’ on the sample's classification. If the expression value of a gene is above or below (for down-regulated genes) a predefined threshold indicating rejection, a single vote is contributed; i.e. the vote score total is incremented by one vote. If the total number of votes (score) from all voting genes exceeds a threshold, the sample is classified as rejections. Score is calculated using the following simple equation of the form:
Score=Σ_{g}V_{g}
where V_{g} is either 1 or 0 depending upon whether the expression of the diagnostic gene or an average of combination of one or more such genes whose expression is correlated to generate a “metagene” is above a threshold (or below for down-regulated genes) and the thresholds are determined to maximize the separation in Score between patients in one class and patients in another class.

Another preferred example of such an algorithm is slightly more complex voting algorithm where highly correlated genes (more than one gene from the same cluster) are combined to improve stability of the signal and instead of a binary yes(1)/no(0) voting scheme, a smoothed transition from 0 to 1 is implemented in the shape of a logistic fit. Raw score is calculated by the following equation:
Score=Σ_{g} exp (α_{g}+β_{g}*C_{T} [g])/(1+exp (α_{g}+β_{g}*C_{T} [g])).
where C₁ . . . C_{Tg} are the logarithmic values of expression of the diagnostic genes in the set or an average of combination of one or more such genes whose expression is correlated to generate a “metagene”, and α₀ . . . αₙ and β₀ . . . βₙ are parameters values (coefficients) determined in such a way so that the separation between the patients in one class and the patients in a second class (i.e., the difference between the average Score values of patients in one class and the average value in the other class) is maximized, and the separation of Score values of patients within a class is minimized.

Another preferred example of such an algorithm is Logistic Regression Algorithm where Logistic regression analysis is applied to classify samples by computing a probability that a sample's true classification is rejection. First a L value is computed by using the equation:
L=Σ_{g}α_{g}C_{Tg}
And then probability is computed that sample is of class rejection (p) as:
p=exp(L)/(1+exp(L)
where C₁ . . . C_{Tg} are the logarithmic values of expression of the diagnostic genes in the set or an average of combination of one or more such genes whose expression is correlated to generate a “metagene”, and α₀ . . . αₙ are parameters values (coefficients) determined in such a way so that the separation between the patients in one class and the patients in a second class (i.e., the difference between the average L values of patients in one class and the average value in the other class) is maximized, and the separation of L values of patients within a class is minimized.

Yet another preferred example of such an algorithm is a linear function of the form:
Score=a₀+a₁C_{T1}+a₂C_{T2}+ . . . +aₙC_{Tn}
where C_{T1} . . . C_{Tn} are the values of expression of the diagnostic genes in the set or an average of combination of one or more such genes whose expression is correlated to generate a “metagene”, and a₀ . . . aₙ are parameters values (coefficients) determined in such a way so that the separation between the patients in one class and the patients in a second class (i.e., the difference between the average Score values of patients in one class and the average value in the other class) is maximized, and the separation of Score values of patients within a class is minimized.

The classes of patients will depend upon what is being monitored in the patients. For example, when transplant rejection is being monitored, then one class would be patients rejecting their transplant and the other class would be patients not rejecting their transplant; when response to an immunosuppressant is being measured, then one class would be patients responding to the immunosuppressant and the other class would be patients not responding to the immunosuppressant; and so on. These coefficients may be determined by use of standard statistical methods such as Discriminant Function Analysis (StatSoft Inc.)

More complicated algorithms may be used when monitoring patients for multiple parameters or measuring gradations within a parameter.

Reagents and Kits

Kits for monitoring the functional status of transplants in patients by detecting the expression levels of a set of diagnostic genes as described above are also disclosed. Each such kit would preferably include instructions in human or machine readable form as well as the reagents typical for the type of assay or assays used to detect expression of the diagnostic genes. These can include, for example, nucleic acid arrays (e.g. cDNA or oligonucleotide arrays), primers and probes for QPCR, antibodies that detect the gene product of each diagnostic gene, each generated to detect the expression profiles of the diagnostic genes. They can also contain reagents used to conduct nucleic acid amplification and detection including, for example, reverse transcriptase, reverse transcriptase primer, a corresponding PCR primer set, a thermostable DNA polymerase, such as Taq polymerase, and a suitable detection reagent(s), such as, without limitation, a scorpion probe, a probe for a fluorescent probe assay, a molecular beacon probe, a single dye primer or a fluorescent dye specific to double-stranded DNA, such as ethidium bromide. Such kits may also contain reagents for detecting gene products of the diagnostic genes such as staining materials specific for a particular gene product, substrates for a particular gene product for enzyme detection or antibodies specific for a particular gene product including accessory components such as buffer, anti-antigenic antibody, detection enzyme and substrate such as Horse Radish Peroxidase or biotin-avidin based reagents.

### EXAMPLES

The following non-limiting examples demonstrate how the methods and compositions disclosed herein may be used and include generation of a simple algorithm to combine the data into a single score. One of skill in the art would recognize that the data may be combined and/or analyzed using any available mathematic formula or algorithm.

### Example 1

### Diagnostic Gene Sets with Voting Algorithms

In this example, the diagnostic gene set determined in the training phase to provide significant separation between rejection and quiescence were used in a simple voting algorithm as discussed above. In this diagnostic gene set, one gene was selected from the Transcription Control Cluster, two diagnostic genes were selected from the Steroid Responsive Gene Cluster, one diagnostic gene was selected from the Heart Failure Cluster, one diagnostic gene was selected from the Early Activation Cluster, one gene was selected from the Cell-Surface Mediated Signaling Cluster, one gene was selected from the Dendritic Cell Maturation Cluster, one gene was selected from the T/B Cell Regulation Cluster, and one additional gene not from any cluster or table was also selected. The diagnostic gene set was based upon maximizing the separation between the rejection samples and quiescent samples (i.e., the average y value of the Rejecters versus the Non-rejecters). Using the test classes of rejection samples and quiescent samples, the initial cutoffs for the voting algorithm were determined by maximizing the separation between the rejection samples and quiescent sample and minimizing the separation within the respective samples.

TABLE 17
Cutoffs for the simple voting algorithm

| Gene | V = 1 if C_{T} |
|---|---|
| HIAN7 | <26.5 |
| ADM | ≧31.8 |
| IL1R2 | ≧34.2 |
| CXCR4-1 | ≧26.3 |
| ITGAM | ≧26.9 |
| DAB1 | ≧29.1 |
| ITGA4 | <27.9 |
| NOTCH1 | <27.6 |
| FLT3LG | <27.2 |

Score is calculated by the equation of simple voting algorithm:
Score=Σ_{g}V_{g} and Samples that have Score>4 were classified as rejecters

In this example, the diagnostic gene set determined in the training phase to provide significant separation between rejection and quiescence were used in a slightly more complex voting algorithm as discussed above. In this diagnostic gene set, two diagnostic genes were selected from the Hematopoiesis Cluster, two diagnostic genes were selected from the Megakaryocyte Cluster, four diagnostic genes were selected from the Steroid Responsive Gene Cluster, two genes were selected from the Inflammatory Cell Recruitment Cluster, one gene was selected from the Cell-Surface Mediated Signaling Cluster, one gene was selected from the T/B Cell Regulation Cluster, one gene was selected from the Transcription Control Cluster, one gene was selected from the Early Activation Cluster, and two additional genes not from any cluster or table were also selected. Further, the expression of two diagnostic genes from the Steroid Responsive Gene Cluster were averaged together as a single “metagene,” the expression of the two diagnostic genes from the Hematopoiesis Cluster were averaged together as a single “metagene,” the expression of the two diagnostic genes from the Inflammatory Cell Recruitment Cluster were averaged together as a single “metagene,” and the expression of two diagnostic genes from the Megakaryocytes Cluster were averaged together as a single “metagene.” The diagnostic gene set and the selection of metagenes was based upon maximizing the separation between the rejection samples and quiescent samples (i.e., the average y value of the Rejecters versus the Non-rejecters). Using the test classes of rejection samples and quiescent samples, the initial coefficients for the voting algorithm were determined by maximizing the separation between the rejection samples and quiescent sample and minimizing the separation within the respective samples.

TABLE 18
Representative coefficients.

| Gene (or metagene) | α | β |
|---|---|---|
| MIR_WDR40A | 54.64 | −0.931 |
| CXCR4 | −46.12 | 1.753 |
| RHOU | 93.88 | −3.147 |
| ITGB7_CBLB | 88.80 | −1.539 |
| FLT3 | −37.90 | 1.182 |
| FLT3LG | 72.63 | 2.645 |
| PF4_G6B | −34.57 | 0.660 |
| ITGA4 | 85.95 | −3.090 |
| ITGAM_S100A9 | −61.30 | 1.244 |
| SIRPB1 | −65.78 | 2.346 |
| TNFSF6 | 43.44 | −1.317 |
| ZNFN1A1 | 144.97 | −5.406 |

Scores are calculated using the equation:
Score=Σ_{g} exp (α_{g}+β_{g}*C_{T} [g])/(1+exp (α_{g}+β_{g}*C_{T} [g])).
where C₁ . . . C_{Tg} are the logarithmic values of expression of the diagnostic genes in the set or an average of combination of one or more such genes whose expression is correlated to generate a “metagene”, and α₀ . . . αₙ and β₀ . . . βₙ are parameters values (coefficients) determined in such a way so that the separation between the patients in one class and the patients in a second class is maximized, and the separation of Score values of patients within a class is minimized as shown in Table 18.

Score obtained from the above equation are mapped from 0 to 40 by the following logistic transformation:
Mapped_Score=40*exp (−4.613+0.825*Score)/(1+exp (−4.613+0.825*Score))

If Mapped_Score≧20, then the samples are classified as belonging to class of Rejecters else samples are classified as Non-rejecters.

### Example 2

### A Diagnostic Gene Set with a Logistic Regression Algorithm

In this example, the diagnostic gene set determined in the training phase to provide significant separation between rejection and quiescence were used in a logistic regression algorithm as discussed above. In this diagnostic gene set, one gene was selected from the Transcription Control Cluster, one diagnostic gene was selected from the Steroid Responsive Gene Cluster, one diagnostic gene was selected from the Hematopoiesis Cluster, one gene was selected from the T/B Cell Regulation Cluster, and two additional genes not from any cluster or table were also selected. The diagnostic gene set was based upon maximizing the separation between the rejection samples and quiescent samples Using the test classes of rejection samples and quiescent samples, the initial coefficients for the logistic regression algorithm were determined by maximizing the separation between the rejection samples and quiescent sample and minimizing the separation within the respective samples. First step is to compute value of L by the following equation:
L=Σ_{g}α_{g}C_{Tg}

where the α_{g} are obtained from the Table 19 and C_{Tg} is the log of expression values of the genes. Once the L is compute, Compute probability that sample is of class rejection (p) as p=exp(L)/(1+exp(L). If the p is more than 0.5 then the sample belongs to the rejection class.

TABLE 19
Coefficients for the logistic regression algorithm

| Gene | α_{G} |
|---|---|
| RHOU | −3.31 |
| MIR | −2.08 |
| FLT3LG | 1.59 |
| ITGAM | 4.38 |
| TNFRSF6 | 4.87 |
| ZNFN1A1 | −5.72 |

### Example 3

### Diagnostic Gene Sets with Linear Algorithms

Various other diagnostic gene sets were designed and tested for use with a linear algorithm. The following example describes three diagnostic gene sets with coefficients for the linear algorithm. The diagnostic gene sets for this example were assembled by selecting particularly informative diagnostic genes, but other diagnostic genes from the clusters may be used as well.

In the first diagnostic gene set, three diagnostic genes were selected from the Steroid Responsive Gene Cluster, two diagnostic genes were selected from the Hematopoiesis Cluster, one diagnostic gene was selected from the T-cell Cluster, one gene was selected from the Cell-Surface Mediated Signaling Cluster, two genes were selected from the Megakaryocytes Cluster, and two additional genes not from any cluster or table were also selected. Further, the expression of three diagnostic genes from the Steroid Responsive Gene Cluster were averaged together as a single “metagene,” the expression of the two diagnostic genes from the Hematopoiesis Cluster were averaged together as a single “metagene,” and the expression of two diagnostic genes from the Megakaryocytes Cluster were averaged together as a single “metagene.” The diagnostic gene set and the selection of metagenes was based upon maximizing the separation between the rejection samples and quiescent samples (i.e., the average y value of the Rejecters versus the Non-rejecters). Using the test classes of rejection samples and quiescent samples, the initial coefficients for the linear algorithm were determined by maximizing the separation between the rejection samples and quiescent sample and minimizing the separation within the respective samples.

Score is calculated using the equation for linear algorithm
Score=a₀+a₁C_{T1}+a₂C_{T2}+ . . . +aₙC_{Tn}
where C_{T1} . . . C_{Tn} are the values of expression of the diagnostic genes in the set or an average of combination of one or more such genes whose expression is correlated to generate a “metagene”, and a₀ . . . aₙ are parameters values (coefficients) determined and described in Table 20.

The scores are further mapped by logistic transformation of the score using the equation
MScore=40*exp (0.234+0.408*S₁)/(1+exp (0.234+0.408*S₁))
to produce a score ranging between 0 and 40 with higher scores being associated with rejection. The algorithm was first applied to the entire training set (36 high-grade rejection and 109 quiescent samples). Using the bootstrap method, the sensitivity and specificity with respect to biopsy of the algorithm were estimated to be 80% and 59%, respectively, with a single, pre-defined threshold (20) for the algorithm score (i.e. a score≧20 is called rejection, otherwise quiescent).

Representative coefficients are shown in table 20.

TABLE 20
Coefficients for the linear algorithm

| | |
|---|---|
| IL1R2_FLT3_ITGAM | 1.41 |
| MIR_WDR40A | −1.64 |
| PDCD1 | −0.84 |
| ITGA4 | −1.34 |
| SEMA7A | −0.74 |
| PF4_G6b | 0.34 |
| ARHU | −0.68 |
| Constant | 105.96 |

In a second diagnostic gene set, one diagnostic gene was selected from the Steroid Responsive Gene Cluster, one diagnostic gene was selected from the Hematopoiesis Cluster, one diagnostic gene was selected from the Dendritic Cell Maturation Cluster, one gene was selected from the Megakaryocytes Cluster, and an additional gene not from any cluster or table was selected. The diagnostic gene set was also based upon maximizing the separation between the rejection samples and quiescent samples Score is calculated using the equation for linear algorithm
Score=a₀+a₁C_{T1}+a₂C_{T2}+ . . . +aₙC_{Tn}
where C_{T1} . . . C_{Tn} are the values of expression of the diagnostic genes in the set or an average of combination of one or more such genes whose expression is correlated to generate a “metagene”, and a₀ . . . aₙ are parameters values (coefficients) determined and described in Table Y.

The scores are further mapped by logistic transformation of the score using the equation
MScore=40*exp (0.234+0.408*S₁)/(1+exp (0.234+0.408*S₁))

to produce a score ranging between 0 and 40 with higher scores being associated with rejection. The algorithm was first applied to the entire training set (36 high-grade rejection and 109 quiescent samples). Using the bootstrap method, the sensitivity and specificity with respect to biopsy of the algorithm were estimated to be 73% and 60%, respectively, with a single, pre-defined threshold (20) for the algorithm score (i.e. a score≧20 is called rejection, otherwise quiescent).

TABLE 21
Coefficients for the linear algorithm

| | |
|---|---|
| FLT3 | 0.95 |
| WDR40A | −1.44 |
| ZFYVE27 | −3.48 |
| PF4 | −0.73 |
| ARHU | −2.86 |
| Constant | 222.51 |

In a third diagnostic gene set, one diagnostic gene was selected from the Steroid Responsive Gene Cluster, one diagnostic gene was selected from the Hematopoiesis Cluster, one diagnostic gene was selected from the Dendritic Cell Maturation Cluster, one gene was selected from the Megakaryocytes Cluster, and an additional gene not from any cluster or table was selected. The diagnostic gene set was also based upon maximizing the separation between the rejection samples and quiescent samples.

Score is calculated using the equation for linear algorithm
Score=a₀+a₁C_{T1}+a₂C_{T2}+ . . . +aₙC_{Tn}
where C_{T1} . . . C_{Tn} are the values of expression of the diagnostic genes in the set or an average of combination of one or more such genes whose expression is correlated to generate a “metagene”, and a₀ . . . aₙ are parameters values (coefficients) determined and described in Table Z.

The scores are further mapped by logistic transformation of the score using the equation
MScore=40*exp (0.234+0.408*S₁)/(1+exp (0.234+0.408*S₁))
to produce a score ranging between 0 and 40 with higher scores being associated with rejection. The algorithm was first applied to training set Using the bootstrap method, the sensitivity and specificity with respect to biopsy of the algorithm were estimated to be 72% and 59%, respectively, with a single, pre-defined threshold (20) for the algorithm score (i.e. a score≧20 is called rejection, otherwise quiescent).

Representative coefficients are shown in table 22.

TABLE 22
Coefficients for the linear algorithm

| | |
|---|---|
| ADM | 1.38 |
| S100A9 | 1.96 |
| ARHU | −2.29 |
| WDR40A | −0.90 |
| BCL6 | −1.66 |
| Constant | 52.64 |

### Example 4

### Patient Study

Patients with recent heart transplants were evaluated with a twenty gene panel selected from the various clusters and with the standard method of taking biopsies from the heart. In this case, the preferred linear algorithm discussed above was generated using two classes of patients: patients showing rejection and patients not showing rejection (or quiescent patients).

Study Design

All patients undergoing heart transplantation at eight transplant centers were eligible for the study. The study was conducted after approval by local Institutional Review Boards and all patients provided written informed consent. The primary study objective was to develop and validate a gene expression blood test that distinguishes acute rejection from quiescence. Additional objectives included correlation of the test to graft dysfunction, drug regimen and CMV infection.

Blood Sampling

Patient samples were processed on the day of surveillance biopsies, upon evaluation for suspected rejection, hospitalization for complications, and suspicion of cytomegalovirus (CMV) infection. Clinical data were collected for each patient encounter.

Endomyocardial Biopsies and Sample Selection

Biopsies were performed by standard techniques and graded by local pathologists and by three independent (“central”) pathologists blinded to clinical information. The biopsy criteria for high-grade rejection were that at least two of four pathologists assigned ISHLT grade≧3A for samples>3 weeks from transplant, transfusion or rejection therapy. Mild rejection samples were defined as grades 1A, 1B or 2 from all 3 central pathologists. Samples designated quiescent were required to be ISHLT grade 0 by three of four readers with no ISHLT grade>1A, no biopsy grade>0 for 3 weeks prior to or 3 weeks after the current sample, no current graft dysfunction, and no biopsy rejection grade≧3A or rejection therapy within the subsequent 3 months. All these criteria were prospectively defined prior to the validation study.

Outcome Measures

Absence of ISHLT grade≧3A by biopsy was the prospectively defined primary outcome measure. Secondary outcome measures included allograft dysfunction defined by pulmonary capillary wedge pressure (PCW)≧20 mmHg.

Expression Profiling

PBMC were isolated from eight mL venous blood using density gradient centrifugation (CPT tubes, Becton-Dickinson). Samples were frozen in lysis buffer (RLT, Qiagen) within two hours of phlebotomy. Total RNA was isolated from each sample (RNeasy, Qiagen) and assessed spectrophotometrically (Spectromax).

Real-Time PCR

PCR primers and probes were designed using PRIMER3 (version 0.9, Whitehead Research Institute). Assays were qualified for inclusion in algorithm development by specificity, linear dynamic range, and efficiency using both human PBMC cDNA and synthetic oligonucleotide templates. For each gene, triplicate 10 μl real-time PCR reactions were performed using FAM-TAMRA probes and standard Taqman reagents and conditions (Applied Biosystems) on cDNA from 5 ng of total RNA. For each sample, expression values, measured by C_{T} (threshold cycle), for a given gene were normalized to a set of empirically identified control genes (18s, GPI, RPLP1, ERCC5, GABPB2, LPPR2) amplified on the same PCR reaction plate. All PCR reactions were run on the ABI 7900HT system

Statistical Analysis—Development of the Clusters

Analysis Microarrays were used only for identification of candidate genes for PCR assay development and validation studies (not to derive and validate classifiers for rejection). Gene selection from microarrays was accomplished by Statistical Analysis of Microarrays (SAM), hierarchical clustering by Cluster 3 and visualization by Java Tree View and non-parametric analysis (Fischer exact). PCR experiment analysis with Student's t-test and median ratios, hierarchical clustering by TreeView and biological relevance derived the final panel of genes for algorithm development.

Several methods were tested for PCR assay algorithm development using this final panel of genes including linear discriminant analysis (StatSoft Inc.), logistic regression (SAS Institute Inc.), Prediction Analysis of Microarrays (PAM), voting (simple, smoothed and layered), TreeNet (Salford Systems), Random Forests, and k-nearest neighbors. The final classifier was developed using linear discriminant analysis as implemented in the “Discriminant Function Analysis” module of Statistica (StatSoft Inc.) in a forward stepwise manner. The classification method was evaluated using cross-validation and bootstrap estimates, an independent test set, robustness to experimental variation, and biological plausibility. All t-tests were at 0.05 (two-sided).

Patient Characteristics

The 700 samples from 249 patients studied were selected from 4917 encounters of the 629 patients in the CARGO study. This subset included all high-grade rejections meeting inclusion criteria. Donor and recipient characteristics were similar to those reported by the United Network for Organ Sharing (UNOS). Most patients received triple therapy with cyclosporine or tacrolimus, mycophenolate mofetil and steroids. Patient characteristics for the rejection and quiescent groups were compared for all experiments and were statistically similar (Table 23).

Correlation of a Real-Time PCR Multi-gene Algorithm Score to Pathological and Clinical Endpoints

As discussed above, the linear discriminant algorithm derived was a combination of expression levels of the informative genes which best distinguished high-grade rejection from quiescence in the training set, plus a set of additional genes for quality control and normalization.

The independent validation set of 270 samples from 172 patients was then tested in a prospective and blinded manner. This set comprised 62 high-grade rejections from 50 patients, 86 mild rejections from 69 patients, and 122 quiescent samples from 83 patients (Table 23). The same score threshold in this independent set yielded sensitivity for high-grade rejection of 76±9% and a specificity of 41±7% as compared to biopsy.

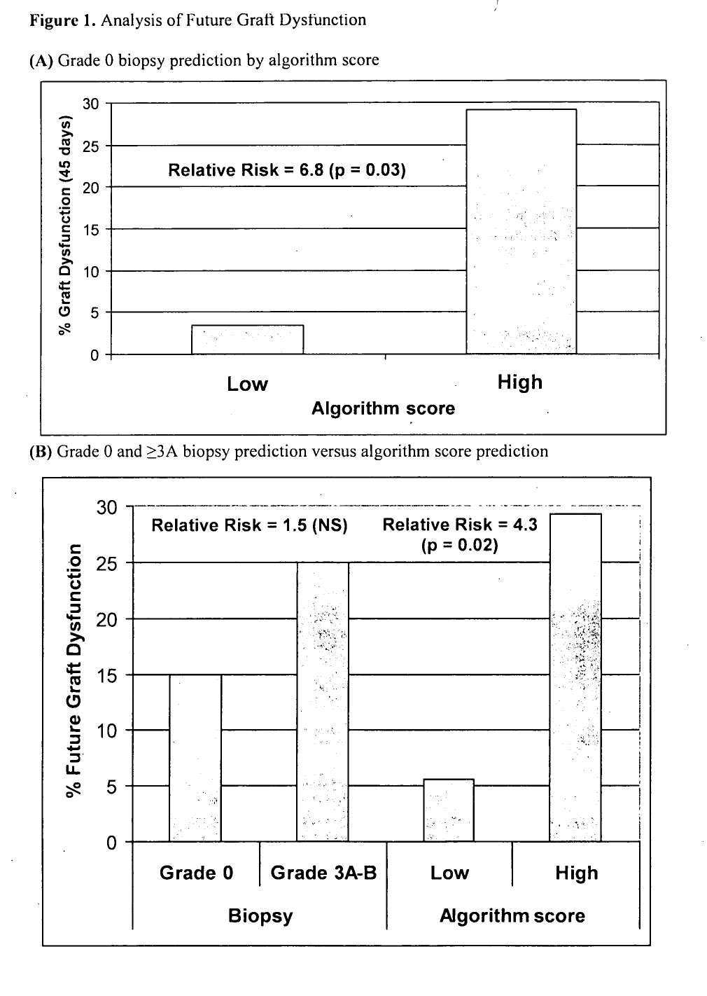

Further refinement of the algorithm took into account time post-transplant, which was observed to be the single most important score-correlated variable. Scores for both rejection and quiescent samples increase with time post-transplant. An important clinical correlate to time post-transplant is corticosteroid dose, which decreases with time post-transplant. Subsequent analysis showed that setting a lower threshold (18.5) for the samples collected within 4 months post-transplant, and a higher threshold (26.5) for the later period, yielded an improved overall specificity of 57% while maintaining an overall sensitivity of 74%, in comparison to biopsy. Within the set of samples collected more than 4 months post-transplant, the higher threshold had a 65% specificity and 75% sensitivity. In post-6 month samples, with a threshold of 28, the sensitivity of the algorithm is 71% and the specificity is 79%. In post-1 year samples, using an even higher threshold of 30, the sensitivity of the algorithm is 80% and the specificity is 78%, in comparison to biopsy (Table 24). In addition to the sensitivity to current biopsy-defined rejection, we explored the ability of algorithm scores to predict graft dysfunction, an important clinical endpoint. In the early post-transplant cohort (<4 months) the high (>18.5) scores identified future graft dysfunction (PCW>20) within 45 days among samples that were grade 0 by biopsy and no current graft dysfunction (FIG. 1). The average scores for those with and without graft dysfunction within 45 days were 24.5 and 15.8, respectively (P=0.011). In addition, the relative risk (RR) for graft dysfunction of patients with a grade 0 biopsy and elevated score (>18.5) was 6.8 compared to those with a low score. Among all patient samples in the early period (n=91) the biopsy result itself did not significantly predict future graft dysfunction (RR=1.5, NS) whereas the score did (RR=4.3, P=0.02).

Discussion

These analyses demonstrate that a molecular test based on changes in PBMC gene expression of genes selected from sixteen Clusters can identify cardiac allograft patients with quiescent alloimmune responses who may not require biopsy. The molecular signatures that differentiate rejection include sixteen discrete clusters of genes that map to specific immune-activation pathways (see tables 1-16).

In this example, real-time PCR was used to develop and validate a multi-gene expression algorithm and assay. This technology is more sensitive than microarrays and can provide a highly reproducible method of assessing gene expression for patient testing. However, in certain embodiments of the present disclosure, microarrays and other techniques for measuring gene expression may be used.

Endomyocardial biopsy has been the standard for diagnosis of rejection in cardiac transplant recipients for decades. Classification by the molecular algorithm derived in this study was found to correlate more closely with grade 3A rejection called by the central versus local pathologists. In addition, grade 2 and 1B cases had lower scores than grade≧3A cases, on average. Through the process of centralized pathology reading, significant variability in the interpretation of biopsies was seen. The maximum concordance for ≦3A rejection between two central pathologists was 77%, and represents the effective limit for our sensitivity performance. The observed sensitivity in the validation study (76+9%) was indistinguishable from this limit and therefore it may prove appropriate to use a threshold at a higher specificity (and lower agreement with biopsy) in clinical practice.

In addition, subendocardial lymphocytic infiltrates (Quilty B lesions) caused significant confusion and over-diagnosis of rejection by biopsy. The central pathologists “downgraded” 60% of local grade 3A and 3B biopsies, of which 42% had Quilty lesions. These findings identify the shortcomings of the pathological grading scheme and are likely to be factors in the discordance observed between molecular test results and biopsy. They suggest that there is an excess of rejection diagnoses in clinical practice, which may lead to the excessive use of immunosuppression and to long-term complications.

In this study biopsy did not correlate with graft dysfunction. In contrast, the algorithm detected future graft dysfunction. The sensitivity of the algorithm score to future graft dysfunction within 45 days for those who are negative by biopsy also suggests that the specificity of gene expression algorithm reported in this study is a lower limit.

Using the 4917 clinical encounters in the CARGO database, the rate of rejection (ISHLT grade≧3A) in stable outpatients seen for a routine biopsy is very low (2.7%). This number is an over-estimate given the finding that local pathologists call 3A rejection at 1.5 times the rate of central pathologists. Rates of graft dysfunction in this outpatient population were 4.3% of patient samples. Stable outpatients who have no signs or symptoms suggestive of rejection, normal graft function echocardiographically and a quiescent algorithm score are a low risk subgroup that could potentially be managed without a biopsy. In this analysis, 63.4% of patients are identified as quiescent and would not need biopsies, assuming graft dysfunction and rejection are independent. In fact, rejection, graft dysfunction and clinical signs and symptoms are not independent which would increase the size of the quiescent patient group. Reduction in the number of biopsies and substitution by a noninvasive test may provide considerable patient benefits and health care expenditure savings.

This approach may also be used to identify patients with low algorithm scores who are over-treated with immunosuppressive drugs and may be candidates for more aggressive weaning. Alternatively, patients may be identified with scores indicating current rejection or impending graft dysfunction who may benefit from augmentation of immunosuppression. Defining the clinical use of this approach to monitor immunosuppression will require further studies.

While this example demonstrates the use of the present methods and compositions with heart transplant monitoring, one of skill in the art would recognize the utility of the present methods and compositions to all other forms of transplant rejection given the commonality of the rejection process for foreign cells, tissue, and organs.

### Example 5

### Predicting Future Rejection Events

The data generated from the patient study described in Example 4 was further analyzed to demonstrate that the diagnostic gene sets and algorithms may be used to predict which patients are more likely to experience rejection.

Longitudinal Analysis of Molecular Algorithm in Patients followed throughout the First Year

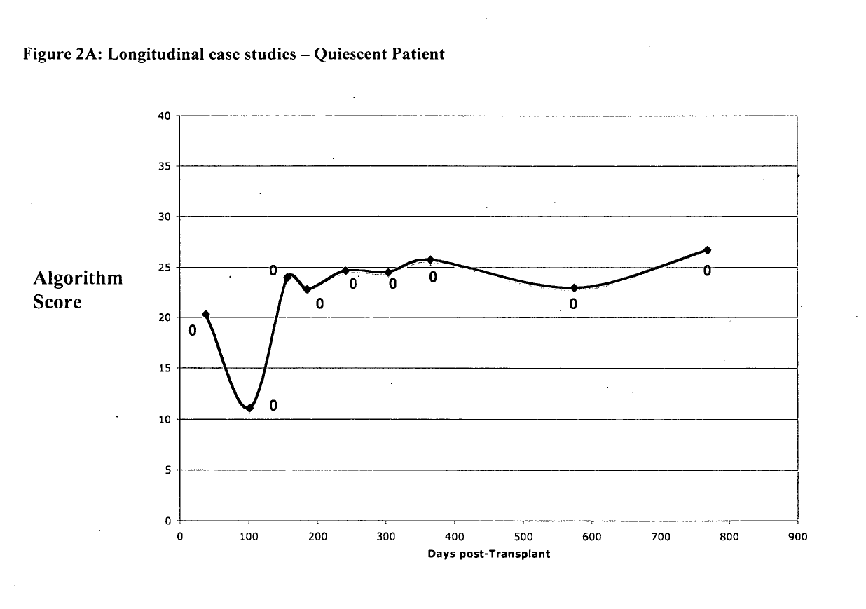

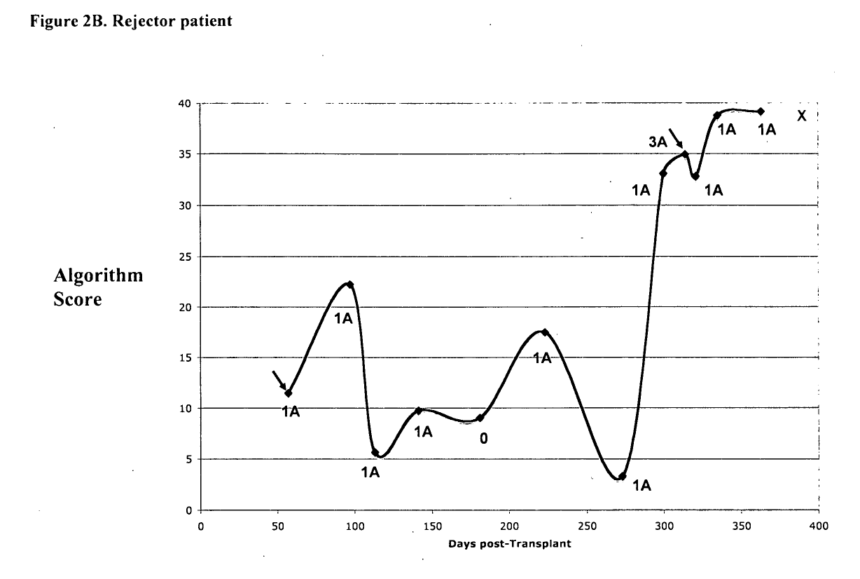

To further demonstrate the utility of the current algorithm, all samples from encounters of 40 patients who were followed for more than 1 year were analyzed. Two illustrative patient profiles are shown in FIGS. 2A and 13 where algorithm score and biopsy grade are indicated for all visits. In the first case (FIG. 2A) a patient showed a benign clinical course throughout the 9 encounters (no biopsy determined 3A rejections) and the algorithm score was below the threshold throughout. In this case the patient could have been managed without multiple biopsies. In the second case (FIG. 2B) the patient shows a more variable clinical course and poor outcome. Here the algorithm score rises above the threshold when the biopsy shows ISHLT grade 1A (no high grade rejection) and remains very high despite rejection therapy, whereas biopsy returns to a lower score. Subsequently the patient died due to multi-organ system failure and sepsis. In this second case it appears that the discordances between algorithm score and biopsy are better explained by the former and might have led to a better clinical treatment paradigm. The longitudinal studies clearly demonstrate that the algorithm is indicative of future rejection as the algorithm score of the patient in the second case increases before the patient began to experience acute rejection as demonstrated by

Statistical Demonstration of the Predictive Value

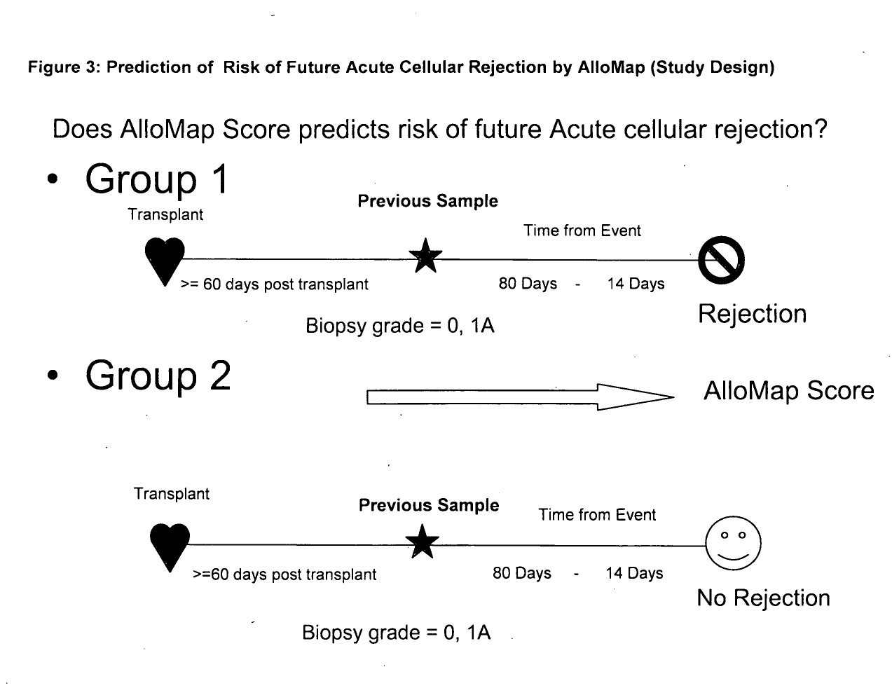

To further demonstrate the statistical significance of the phenomenon, a double-blind prospective multi-center study of archive samples from CARGO was performed. The study was designed to ask if high algorithm scores are associated with significantly increased likelihood of near-term acute rejection, defined as an ISHLT biopsy grade of 3A or greater in the future. Preceding Samples (biopsy grade 0 or 1A) before High grade Rejection or No Rejection were prospectively selected from the existing CARGO database and both groups were carefully balanced in terms of Days Post Transplant, Steroid dose and time interval before an event (See FIG. 3). Algorithm scores were obtained prospectively in a blinded manner using the same algorithm used in Example 4, which was based on RT-PCR measurements for multiple genes for each study sample. Sample numbers were chosen so that study was powered. Forty samples from patients which went on to have high grade rejection and eighty samples from patients which did not have rejection were used for this study. Samples that were included in the training of the algorithm were not selected. Samples labels were encrypted and the lab technicians were blinded to the clinical data associated with each sample, minimizing any operator bias. T-test, a parametric test, and Mann-Whitney, a non-parametric test, were performed on the algorithm scores obtained from both the groups. T-test p-value obtained was 0.01; Mann-Whitney p-value obtained was 0.0048 and the Area under curve for ROC=0.66±0.05 with a Z score of 2.8. Thus proving that high algorithm scores are significantly associated with near-term rejection (14-80) days. The relative risk for patients with scores above the median for future rejection within the 14-80 day time period was 1.85 (CI 1.06-3.23).

### Example 6

### Preparation of PBMC

The following protocol represents a typical procedure of the preparation of peripheral blood mononucleocyte cells that may be profiled with the probe sets for the monitoring of the functional status of transplants in patients.

Eight ml of blood is collected into the Vacutainer CPT tube and the CPT tube is inverted 10-15 times to fully mix blood with tube contents. The CPT tube is centrifuged at 3400 rpm (1750×g) for 15 minutes at room temperature (18 to 25° C.) in a centrifuge. For best results, the next two steps immediately following centrifugation are performed and draw to freezer time should not be more than 2 hours. The CPT tube is inverted 10-15 times to resuspend the separated mononuclear cells into the plasma. The plasma/mononuclear cell mixture, the layer above the gel, from the CPT tubes is poured into the labeled centrifuge tube containing phosphate buffered saline. The phosphate buffered saline tube is capped and inverted 10-15 times to mix and centrifuged for 5 minutes at 3400 rpm (1750×g) in the CL-2 centrifuge. The mononuclear cells form a pellet in the bottom of the tube. The supernatant is poured off and discarded. Care is taken to discard as much supernatant as possible by touching the rim of the tube to a paper towel or gauze pad to get the last drop. The cell pellet is then lysed by suspending it in LyseDx by vigorously pipeting the cell pellet and the LyseDx up and down until the cells have completely disappeared and the lysate is clear. LyseDx contains beta-mercaptoethanol and guanidinium thiocyanate as per specifications in (RNeasy® Mini Handbook, Third Edition, Qiagen, Valencia Calif., June 2001). When lysis is complete, tighten the cap on the centrifuge tube and freeze at −15° C. or colder until ready to ship or assay.

### Example 6

### Quality Controls

The following protocols represent a typical procedures to verify that the reagents are suitable for use in profiling a sample from a patient by verifying the quality and reproducibility of past results.

RNA Purification

To ensure that each new lot of reagents or new shipments of current lots used for purification of RNA meet minimum performance criteria, quality control testing of Qiagen RNeasy kits is done. Qiagen RNeasy RNA Purification kit and the Qiagen RNase-Free DNase Set used for purification of RNA are purchased separately. A kit is considered expired after 9 months from the date received. This has been determined by studying stability data.

Quality Control of New Kit is determined by testing CPT lysate from one donor using the new shipment of reagents in parallel with the old RNA Purification Kit currently in use. The new lot is considered approved if it meets the following Evaluation criteria:
1. In 2 of the 3 lysates tested the yield from the new lot must be ≧70% of the yield from the current lot.
2. The A260/A280 ratio of the new lot or shipment must be within 1.5-3.5 for all lysates that pass the yield test.
3. For each assay, the absolute difference between the current lot C_{T}s and the new lot (or shipment) C_{T}s must be ≦0.7.
4. For each donor the average absolute difference of all assays must be ≦0.5 CT 5) Over all donors, there must not be a directional shift to the CT differences. A directional shift is defined as ≧75% or ≦25% of C_{T} differences >0.

The concentration of LTP is adjusted so that the Ct for the LTP assay must be 22±2 which comes to 6.67 pM.

cDNA Synthesis:

Quality control testing is performed on new lots and new shipments of current lots of cDNA synthesis reagents before the reagents are used.

A cDNA Synthesis lot includes Superscript II Reverse Transcriptase, 5× First Strand Buffer, 100 mM DTT, Oligo dT, Random Hexamer, RNaseOUT, dNTPs and RNase H. The components are purchased as individual reagents and grouped to form a single lot of reagents. Quality control is performed on the lot and no reagent substitutions may be made to the lot for use in routine testing. The expiration date for the lot is the earliest expiration date of any of the lot components. Quality Control of New Kit is determined by testing RNA from 2 different donors (patients or donors) and the current control sample using the new lot of reagents in parallel with the cDNA lot currently in use.

The new lot is considered approved if it meets the following Evaluation criteria:
1. For each assay, the absolute difference between the current lot C_{T}s and the new lot (or shipment) C_{T}s must be ≦0.7.
2. For each donor the average absolute difference of all assays must be ≦0.5 C_{T}
3. Over all donors, there must not be a directional shift to the C_{T} differences. A directional shift is defined as ≧75% or ≦25% of C_{T} differences >0.

QPCR Assay:

Final concentrations of different reagents are as follows:

600 nM Primers

300 nM Probe

1× Universal Master Mix from ABI

0.5 ng cDNA

A synthetic known amount of lipid transfer protein (LTP) template is used as a PCR control on every plate of QPCR. This is a plant gene and is not supposed to have any expression in human sample.

Sequence of LTP:

| | |
|---|---|
| (5′-TGCTTACAGTCCGCTGCAAAAGGGGTTAATCCAAG | SEQ ID NO:1 |
| TCTAGCCTCTGGCCTTCCTGGAAAGTGCGGTGTTAGCAT | |
| CCCCTATCCCATCTCC-3′) | |

### Example 7

### MicroArray Clustering

In this example, sixty-eight mRNA samples obtained from patients (both rejectors and non-rejectors) were screened on microarrays to determine the expression levels of genes in rejectors as compared to non-rejectors. The microarrays largely confirmed the assignment of clusters of genes with correlated expression patterns.

Microarray

Microarray experiments were performed on Agilent Human Whole Genome chips using the Agilent specific standard operating procedures provided with the Agilent Genome chips. Microarray data were processed by Agilent FE plug-in and loaded into GeneSpring software. Non-normalized processed raw signal data was used in the data analysis by GeneSpring. GeneSpring is collection of software programs used for desktop expression data analysis. The Agilent Genome chips include 41,000 genes on the chip. Following steps were carried out in analyzing the expression data with GeneSpring:

Filtering:
1) Flags were present on at least 54 of the 68 (80%) microarray chips.
2) Processed raw signal data was greater than or equal to 100 on at least 54 of the 68 (80%) microarray chips.
3) Number of genes after filtering: 28,457

Clustering:
1) After filtering, 28,457 genes were clustered by K-means clustering. The following parameters were used: Number of clusters 160, Similarity Measure Pearson Correlation.
2) If clusters contained at least 1 of the probes corresponding to main gene(s) in the original clusters then all genes in the clusters were combined for another round of clustering as described above.
3) After 10 rounds of clustering, 385 genes from clusters containing at least 1 original member were kept. Finally each of these clusters were clustered using hierarchical clustering with average linkage method where the Pearson correlation was used as the similarity metric to derive the average correlation between the clusters.

The final clusters are as shown on Tables 1 through Table 16.

TABLE 1
the Cell-Surface Mediated Signaling Cluster

| correlation | | | gene | |
|---|---|---|---|---|
| coefficient | subcluster | source | symbol | annotation |
| 0.49 | (SC 1) | M | MGC14560 | Homo sapiens protein x 0004 (MGC14560), mRNA |
| | (SC 1) | M | ZNFN1A1 | Homo sapiens zinc finger protein, subfamily 1A, 1 |
| | | | | (Ikaros) (ZNFN1A1), mRNA |
| | (SC 1) | M | ZNF274 | Homo sapiens zinc finger protein 274 (ZNF274), |
| | | | | transcript variant ZNF274c, mRNA |
| | (SC 1) | M | SSR3 | Homo sapiens signal sequence receptor, gamma |
| | | | | (translocon-associated protein gamma) (SSR3), |
| | | | | mRNA |
| | (SC 1) | M | IFI16 | Homo sapiens interferon, gamma-inducible protein |
| | | | | 16 (IFI16), mRNA |
| | (SC 1) | M | C6orf33 | Homo sapiens chromosome 6 open reading frame 33 |
| | | | | (C6orf33), mRNA |
| | (SC 1) | M | MAN2B2 | Homo sapiens mannosidase, alpha, class 2B, |
| | | | | member 2 (MAN2B2), mRNA |
| | (SC 1) | M | NF2 | Homo sapiens neurofibromin 2 (bilateral acoustic |
| | | | | neuroma) (NF2), transcript variant 2, mRNA |
| | (SC 1) | M | LOC123169 | Homo sapiens senescence downregulated leo1-like |
| | | | | (LOC123169), mRNA |
| | (SC 1) | M | FLJ14825 | Homo sapiens hypothetical protein FLJ14825 |
| | | | | (FLJ14825), mRNA |
| | (SC 1) | M | EGLN1 | Homo sapiens egl nine homolog 1 (C. elegans) |
| | | | | (EGLN1), mRNA |
| | (SC 1) | M | C14orf92 | Homo sapiens T-cell receptor alpha delta locus from |
| | | | | bases 1 to 250529 (section 1 of 5) of the Complete |
| | | | | Nucleotide Sequence. |
| | (SC 1) | M | TTC17 | Homo sapiens tetratricopeptide repeat domain 17 |
| | | | | (TTC17), mRNA |
| | (SC 1) | M | FLJ20257 | Homo sapiens hypothetical protein FLJ20257 |
| | | | | (FLJ20257), mRNA |
| | (SC 1) | M | METTL3 | Homo sapiens methyltransferase like 3 (METTL3), |
| | | | | mRNA |
| | (SC 1) | M | FTSJ3 | Homo sapiens FtsJ homolog 3 (E. coli) (FTSJ3), |
| | | | | mRNA |
| | (SC 1) | M | COPS5 | Homo sapiens COP9 constitutive photomorphogenic |
| | | | | homolog subunit 5 (Arabidopsis) (COPS5), mRNA |
| | (SC 1) | M | GARS | Homo sapiens glycyl-tRNA synthetase (GARS). |
| | | | | mRNA |
| | (SC 1) | M | N/A | Human chromosome 14 DNA sequence BAC R- |
| | | | | 182E21 of library RPCI-11 from chromosome 14 of |
| | | | | Homo sapiens (Human), complete sequence. |
| | | | | CNS06C8K (Agilent A_24_P270424prop |
| | (SC 1) | M | FLJ20257 | Homo sapiens hypothetical protein FLJ20257 |
| | | | | (FLJ20257), mRNA |
| | (SC 1) | M | FYB | Homo sapiens FYN binding protein (FYB-120/130) |
| | | | | (FYB), mRNA |
| | (SC 1) | M | STK10 | Homo sapiens serine/threonine kinase 10 (STK10), |
| | | | | mRNA |
| | (SC 1) | M | RHOU | Homo sapiens ras homolog gene family, member U |
| | | | | (RHOU), mRNA |
| | (SC 1) | M | N/A | Homo sapiens mRNA for eukaryotic translation |
| | | | | initiation factor 4E member 2 variant protein - |
| | | | | 24302885 (Agilent probe A_24_P934755) |
| | (SC 1) | M | FLJ20519 | Homo sapiens hypothetical protein FLJ20519 |
| | | | | (FLJ20519), mRNA |
| | (SC 1) | M | RAB43 | member RAS oncogene family |
| | (SC 1) | M | RPS6KA3 | Homo sapiens ribosomal protein S6 kinase, 90 kDa, |
| | | | | polypeptide 3 (RPS6KA3), mRNA |
| | SC 1 | P | CD47 | CD47 antigen (Rh-related antigen, integrin- |
| | | | | associated signal transducer |
| | SC 1 | P | FCGR3A | Fc fragment of IgG, low affinity IIIa, receptor for |
| | | | | (CD16) |
| | SC 1 | P | FCGR3B | Fc fragment of IgG, low affinity IIIb, receptor for |
| | | | | (CD16) |
| | SC 1 | P | PRDX4 | peroxiredoxin 4 |
| | SC 1 | B | ITGA4 | Homo sapiens integrin, alpha 4 (antigen CD49D, |
| | | | | alpha 4 subunit of VLA-4 receptor) (ITGA4), mRNA |
| | SC 9 | B | ZNFN1A1 | Homo sapiens zinc finger protein, subfamily 1A, 1 |
| | | | | (Ikaros) (ZNFN1A1), mRNA |
| | SC 9 | B | hIAN7 | Homo sapiens immune associated nucleotide |
| | | | | (hIAN7), mRNA |

TABLE 2
the Inflammation cluster

| correlation | | | gene | |
|---|---|---|---|---|
| coefficient | subcluster | source | symbol | annotation |
| 0.59 | (SC 2) | M | ACSL1 | Homo sapiens acyl-CoA synthetase long-chain family |
| | | | | member 1 (ACSL1), mRNA |
| | (SC 2) | M | ORM1 | Homo sapiens orosomucoid 1 (ORM1), mRNA |
| | (SC 2) | M | CEBPE | Homo sapiens CCAAT/enhancer binding protein |
| | | | | (C/EBP), epsilon (CEBPE), mRNA |
| | (SC 2) | M | CA4 | Homo sapiens carbonic anhydrase IV (CA4), mRNA |
| | (SC 2) | M | SLC22A16 | Homo sapiens solute carrier family 22 (organic cation |
| | | | | transporter), member 16 (SLC22A16), mRNA |
| | (SC 2) | M | S100A12 | Homo sapiens S100 calcium binding protein A12 |
| | | | | (calgranulin C) (S100A12), mRNA |
| | (SC 2) | M | LIN7A | Homo sapiens lin-7 homolog A(C. elegans) (LIN7A), |
| | | | | mRNA |
| | (SC 2) | M | N/A | Agilent probe (A_32_P100109) Homo sapiens mRNA |
| | | | | for RalBP1 associated Eps domain containing protein |
| | | | | 2 variant protein |
| | (SC 2) | M | CD24 | Homo sapiens CD24 antigen (small cell lung |
| | | | | carcinoma cluster 4 antigen) (CD24), mRNA |
| | SC 2 | P | CLC | Charcot-Leyden crystal protein |
| | SC 2 | P | MME | matrix metalloproteinase 12 (macrophage elastase) |
| | SC 3 | B | ITGAM | Homo sapiens integrin, alpha M (complement |
| | | | | component receptor 3, alpha; also known as CD11b |
| | | | | (p170), macrophage antigen alpha polypeptide) |
| | | | | (ITGAM), mRNA |
| | SC 3 | B | S100A9 | Homo sapiens S100 calcium binding protein A9 |
| | | | | (calgranulin B) (S100A9), mRNA |
| | SC 2 | B | MMP9 | Homo sapiens matrix metalloproteinase 9 (gelatinase |
| | | | | B, 92 kDa gelatinase. 92 kDa type IV collagenase) |
| | | | | (MMP9), mRNA |

TABLE 3
the Steroid Responsive Gene Cluster

| correlation | | | | |
|---|---|---|---|---|
| coefficient | subcluster | source | gene symbol | annotation |
| 0.39 | (SC 3) | M | TPST1 | Homo sapiens tyrosylprotein sulfotransferase 1 |
| | | | | (TPST1), mRNA |
| | (SC 3) | M | CPM | Homo sapiens carboxypeptidase M (CPM), |
| | | | | transcript variant 2, mRNA |
| | (SC 3) | M | CXCL9 | Homo sapiens chemokine (C—X—C motif) ligand 9 |
| | | | | (CXCL9), mRNA |
| | (SC 3) | M | Z39IG | Homo sapiens Ig superfamily protein (Z39IG), |
| | | | | mRNA |
| | (SC 3) | M | CEBPD | Homo sapiens CCAAT/enhancer binding protein |
| | | | | (C/EBP), delta (CEBPD), mRNA |
| | (SC 3) | M | CD163 | Homo sapiens CD163 antigen (CD163), transcript |
| | | | | variant 2, mRNA |
| | (SC 3) | M | CXCL10 | Homo sapiens chemokine (C—X—C motif) ligand 10 |
| | | | | (CXCL10), mRNA |
| | (SC 3) | M | DKFZP434B044 | Homo sapiens hypothetical protein |
| | | | | DKFZp434B044 (DKFZP434B044), mRNA |
| | SC 3 | P | FPRL1 | formyl peptide receptor-like 1 |
| | SC 3 | P | S100A8 | S100 calcium binding protein A8 (calgranulin A) |
| | SC 3 | P | NFE2 | nuclear factor (erythroid-derived 2), 45 kDa |
| | SC 3 | B | IL18 | Homo sapiens interleukin 18 (interferon-gamma- |
| | | | | inducing factor) (IL18), mRNA |
| | SC 3 | B | IL1R2 | Homo sapiens interleukin 1 receptor, type II |
| | | | | (IL1R2), transcript variant 2, mRNA |
| | SC 3 | B | FLT3 | Homo sapiens fms-related tyrosine kinase 3 |
| | | | | (FLT3), mRNA |
| | SC 3 | B | CPM | Homo sapiens carboxypeptidase M (CPM), |
| | | | | transcript variant 2, mRNA |

TABLE 4
the Early Activation Cluster

| correlation | | | gene | |
|---|---|---|---|---|
| coefficient | subcluster | source | symbol | annotation |
| 0.521 | SC 4 | P | CXCR4 | C—X—C type |
| | | | | chemokine Receptor 4 |
| | SC 4 | P | CD69 | early T-cell activation |
| | | | | antigen |
| | SC 4 | P | TNFAIP3 | tumor necrosis factor, |
| | | | | alpha-induced protein 3 |

TABLE 5
the Heart Failure Cluster

| correlation | | | gene | |
|---|---|---|---|---|
| coefficient | subcluster | source | symbol | annotation |
| 0.34 | SC 5 | P | ADM | adrenomedullin |
| | SC 5 | P | HMOX1 | heme oxygenase |
| | | | | (decycling) 1 |
| | SC 5 | P | ICAM1 | intercellular adhesion |
| | | | | molecule 1 (CD54), |
| | | | | human rhinovirus receptor |
| | SC 5 | P | TYROBP | TYRO protein tyrosine |
| | | | | kinase binding protein |

TABLE 6
the Hematopoiesis Cluster

| correlation | | | | |
|---|---|---|---|---|
| coefficient | subcluster | source | Gene Symbol | Annotation |
| 0.66 | (SC 6) | M | KRT1 | Homo sapiens keratin 1 (epidermolytic |
| | | | | hyperkeratosis) (KRT1), mRNA |
| | (SC 6) | M | FAM46C | Homo sapiens family with sequence similarity |
| | | | | 46, member C (FAM46C), mRNA |
| | (SC 6) | M | SLC6A8 | Homo sapiens solute carrier family 6 |
| | | | | (neurotransmitter transporter, creatine), |
| | | | | member 8 (SLC6A8), mRNA |
| | (SC 6) | M | EPB41 | Homo sapiens erythrocyte membrane protein |
| | | | | band 4.1 (elliptocytosis 1, RH-linked) (EPB41), |
| | | | | transcript variant 3, mRNA |
| | (SC 6) | M | DKFZp686N09198 | Homo sapiens mRNA; cDNA |
| | | | | DKFZp686N09198 (from clone |
| | | | | DKFZp686N09198); complete cds. |
| | (SC 6) | M | N/A | full-length cDNA clone CS0DJ008YP03 of T |
| | | | | cells (Jurkat cell line) Cot 10-normalized of |
| | | | | Homo sapiens (human). [CR600106] |
| | (SC 6) | M | HBQ1 | Homo sapiens hemoglobin, theta 1 (HBQ1), |
| | | | | mRNA |
| | (SC 6) | M | FLJ32009 | Homo sapiens hypothetical protein FLJ32009 |
| | | | | (FLJ32009), mRNA |
| | (SC 6) | M | HBG2 | Homo sapiens hemoglobin, gamma G (HBG2), |
| | | | | mRNA |
| | (SC 6) | M | HBG1 | Homo sapiens hemoglobin, gamma A (HBG1), |
| | | | | mRNA |
| | (SC 6) | M | C20orf108 | Homo sapiens chromosome 20 open reading |
| | | | | frame 108 (C20orf108), mRNA |
| | (SC 6) | M | SELENBP1 | Homo sapiens selenium binding protein 1 |
| | | | | (SELENBP1), mRNA |
| | (SC 6) | M | AE1 | Human anion exchanger (AE1) gene, exons 1-20. |
| | (SC 6) | M | LAMA3 | Homo sapiens laminin, alpha 3 (LAMA3), |
| | | | | transcript variant 1, mRNA |
| | (SC 6) | M | ALS2CR2 | Homo sapiens amyotrophic lateral sclerosis 2 |
| | | | | (juvenile) chromosome region, candidate 2 |
| | | | | (ALS2CR2), mRNA |
| | (SC 6) | M | GYPB | Homo sapiens glycophorin B (includes Ss |
| | | | | blood group) (GYPB), mRNA |
| | (SC 6) | M | N/A | Homo sapiens , clone IMAGE: 5262833, mRNA. |
| | (SC 6) | M | NFIX | Homo sapiens chromosome 19 cosmid |
| | | | | R34714, complete sequence. |
| | (SC 6) | M | N/A | Agilent Probe (A_32_P161836) |
| | (SC 6) | M | N/A | Homo sapiens chromosome 19 clone LLNL- |
| | | | | R_266C8, complete sequence. |
| | (SC 6) | M | ALAS2 | Homo sapiens aminolevulinate, delta-, |
| | | | | synthase 2 (sideroblastic/hypochromic |
| | | | | anemia) (ALAS2), nuclear gene encoding |
| | | | | mitochondrial protein, mRNA |
| | (SC 6) | M | HBB | Homo sapiens hemoglobin, beta (HBB), |
| | | | | mRNA |
| | SC 6 | P | MIR | cellular modulator of immune recognition |
| | SC 6 | P | MKRN1 | makorin, ring finger protein, 1 RNF61 |
| | SC 6 | P | RNF10 | ring finger protein 10, RIE2, KIAA0262 |
| | SC 6 | P | MSCP | MSCP-like |
| | SC 6 | P | BPGM | 2,3-bisphosphoglycerate mutase |
| | SC 6 | B | EPB42 | Homo sapiens erythrocyte membrane protein |
| | | | | band 4.2 (EPB42), mRNA |
| | SC 6 | B | WDR40A | Homo sapiens WD repeat domain 40A |
| | | | | (WDR40A), mRNA |
| | SC 6 | B | HBA1 | Homo sapiens hemoglobin, alpha 1 (HBA1), |
| | | | | mRNA |
| | SC 6 | B | HBG2 | Homo sapiens hemoglobin, gamma G (HBG2), |
| | | | | mRNA |
| | SC 6 | B | ALAS2 | Homo sapiens aminolevulinate, delta-, |
| | | | | synthase 2 (sideroblastic/hypochromic |
| | | | | anemia) (ALAS2), nuclear gene encoding |
| | | | | mitochondrial protein, mRNA |

TABLE 7
the Megakaryocytes Cluster

| correlation | | | gene | |
|---|---|---|---|---|
| coefficient | subcluster | source | symbol | annotation |
| 0.60 | (SC 7) | M | SH3BGRL2 | Homo sapiens SH3 domain binding glutamic acid- |
| | | | | rich protein like 2 (SH3BGRL2), mRNA |
| | (SC 7) | M | KIF3C | Homo sapiens kinesin family member 3C (KIF3C), |
| | | | | mRNA |
| | (SC 7) | M | GUCY1B3 | Homo sapiens guanylate cyclase 1, soluble, beta |
| | | | | 3 (GUCY1B3), mRNA |
| | (SC 7) | M | ARHGEF12 | Homo sapiens Rho guanine nucleotide exchange |
| | | | | factor (GEF) 12 (ARHGEF12), mRNA |
| | (SC 7) | M | PCSK6 | Homo sapiens proprotein convertase |
| | | | | subtilisin/kexin type 6 (PCSK6), transcript variant |
| | | | | 2, mRNA |
| | (SC 7) | M | IMP-3 | Homo sapiens IGF-II mRNA-binding protein 3 |
| | | | | (IMP-3), mRNA |
| | (SC 7) | M | PRTFDC1 | Homo sapiens phosphoribosyl transferase domain |
| | | | | containing 1 (PRTFDC1), mRNA |
| | (SC 7) | M | TPM1 | Human skeletal muscle alpha-tropomyosin (hTM- |
| | | | | alpha) mRNA, 3′ end. |
| | (SC 7) | M | MYL9 | Homo sapiens myosin, light polypeptide 9, |
| | | | | regulatory (MYL9), transcript variant 2, mRNA |
| | (SC 7) | M | PTGS1 | Homo sapiens prostaglandin-endoperoxide |
| | | | | synthase 1 (prostaglandin G/H synthase and |
| | | | | cyclooxygenase) (PTGS1), transcript variant 2, |
| | | | | mRNA |
| | (SC 7) | M | ABLIM3 | Homo sapiens actin binding LIM protein family, |
| | | | | member 3 (ABLIM3), mRNA |
| | (SC 7) | M | AGPAT1 | Homo sapiens 1-acylglycerol-3-phosphate O- |
| | | | | acyltransferase 1 (lysophosphatidic acid |
| | | | | acyltransferase, alpha) (AGPAT1), transcript |
| | | | | variant 1, mRNA |
| | (SC 7) | M | ANKRD9 | Homo sapiens ankyrin repeat domain 9 |
| | | | | (ANKRD9), mRNA |
| | (SC 7) | M | TPM1 | Homo sapiens tropomyosin 1 (alpha) (TPM1), |
| | | | | mRNA |
| | (SC 7) | M | PCSK6 | Homo sapiens proprotein convertase |
| | | | | subtilisin/kexin type 6 (PCSK6), transcript variant |
| | | | | 6, mRNA |
| | (SC 7) | M | MGC50844 | Homo sapiens hypothetical protein MGC50844 |
| | | | | (MGC50844), mRNA |
| | (SC 7) | M | PARVB | Homo sapiens parvin, beta (PARVB), mRNA |
| | (SC 7) | M | NID67 | Homo sapiens putative small membrane protein |
| | | | | NID67 (NID67), mRNA |
| | (SC 7) | M | | Agilent Probe (A_23_P421843) AK095809 Homo |
| | | | | sapiens cDNA FLJ38490 fis, clone |
| | | | | FEBRA2023764, weakly similar to Rattus |
| | | | | norvegicus neurabin mRNA. |
| | (SC 7) | M | HSPC159 | Homo sapiens HSPC159 protein (HSPC159). |
| | | | | mRNA |
| | (SC 7) | M | GAS2L1 | Homo sapiens growth arrest-specific 2 like 1 |
| | | | | (GAS2L1), transcript variant 3, mRNA |
| | (SC 7) | M | HRASLS | Homo sapiens HRAS-like suppressor (HRASLS), |
| | | | | mRNA |
| | (SC 7) | M | LOC51257 | Homo sapiens hypothetical protein LOC51257 |
| | | | | (LOC51257), mRNA |
| | (SC 7) | M | LOC201191 | Homo sapiens hypothetical protein LOC201191 |
| | | | | (LOC201191), mRNA |
| | (SC 7) | M | PCSK6 | Homo sapiens proprotein convertase |
| | | | | subtilisin/kexin type 6 (PCSK6), transcript variant |
| | | | | 6, mRNA |
| | (SC 7) | M | SH3BGRL2 | Homo sapiens SH3 domain binding glutamic acid- |
| | | | | rich protein like 2 (SH3BGRL2), mRNA |
| | (SC 7) | M | CXCL5 | Homo sapiens chemokine (C—X—C motif) ligand 5 |
| | | | | (CXCL5), mRNA |
| | (SC 7) | M | N/A | Agilent Probe (A_24_P315256) |
| | (SC 7) | M | DDEF2 | Homo sapiens development and differentiation |
| | | | | enhancing factor 2 (DDEF2), mRNA |
| | (SC 7) | M | MGC50844 | Homo sapiens hypothetical protein MGC50844 |
| | | | | (MGC50844), mRNA |
| | (SC 7) | M | PTGS1 | Homo sapiens prostaglandin-endoperoxide |
| | | | | synthase 1 (prostaglandin G/H synthase and |
| | | | | cyclooxygenase) (PTGS1), transcript variant 2, |
| | | | | mRNA |
| | (SC 7) | M | ITGA2B | Homo sapiens integrin, alpha 2b (platelet |
| | | | | glycoprotein IIb of IIb/IIIa complex, antigen |
| | | | | CD41B) (ITGA2B), mRNA |
| | (SC 7) | M | GAS2L1 | Homo sapiens growth arrest-specific 2 like 1 |
| | | | | (GAS2L1), transcript variant 3, mRNA |
| | (SC 7) | M | TPM1 | tropomyosin 1 (alpha) |
| | (SC 7) | M | MGC50844 | Homo sapiens hypothetical protein MGC50844 |
| | | | | (MGC50844), mRNA |
| | (SC 7) | M | LOC387821 | PREDICTED: Homo sapiens hypothetical |
| | | | | LOC387821 (LOC387821), mRNA |
| | (SC 7) | M | CKLFSF5 | Homo sapiens chemokine-like factor super family |
| | | | | 5 (CKLFSF5), transcript variant 2, mRNA |
| | (SC 7) | M | RAB27B | Homo sapiens RAB27B, member RAS oncogene |
| | | | | family (RAB27B), mRNA |
| | (SC 7) | M | GNG11 | Homo sapiens guanine nucleotide binding protein |
| | | | | (G protein), gamma 11 (GNG11), mRNA |
| | (SC 7) | M | PDLIM1 | Homo sapiens PDZ and LIM domain 1 (elfin) |
| | | | | (PDLIM1), mRNA |
| | (SC 7) | M | MAX | Homo sapiens MAX protein (MAX), transcript |
| | | | | variant 6, mRNA |
| | (SC 7) | M | ALOX12 | Homo sapiens arachidonate 12-lipoxygenase |
| | | | | (ALOX12), mRNA |
| | (SC 7) | M | MGC13057 | Homo sapiens hypothetical protein MGC13057 |
| | | | | (MGC13057), mRNA |
| | (SC 7) | M | C19orf33 | Homo sapiens chromosome 19 open reading |
| | | | | frame 33 (C19orf33), mRNA |
| | (SC 7) | M | N/A | A_23_P210060Homo sapiens mRNA; cDNA |
| | | | | DKFZp686I15210 (from clone DKFZp686I15210) |
| | (SC 7) | M | N/A | A_23_P210330Homo sapiens HSPC159 protein, |
| | | | | mRNA (cDNA clone MGC: 33751 |
| | | | | IMAGE: 5301908), complete cds |
| | (SC 7) | M | LIMS1 | Homo sapiens LIM and senescent cell antigen-like |
| | | | | domains 1 (LIMS1), mRNA |
| | (SC 7) | M | ARHGAP6 | Homo sapiens Rho GTPase activating protein 6 |
| | | | | (ARHGAP6), transcript variant 1, mRNA |
| | (SC 7) | M | GP1BB | Homo sapiens glycoprotein lb (platelet), beta |
| | | | | polypeptide (GP1BB), mRNA |
| | (SC 7) | M | PTCRA | Homo sapiens pre T-cell antigen receptor alpha |
| | | | | (PTCRA), mRNA |
| | (SC 7) | M | ELOVL7 | Homo sapiens ELOVL family member 7, |
| | | | | elongation of long chain fatty acids (yeast) |
| | | | | (ELOVL7), mRNA |
| | (SC 7) | M | C19orf33 | Homo sapiens chromosome 19 open reading |
| | | | | frame 33 (C19orf33), mRNA |
| | (SC 7) | M | GNAZ | Homo sapiens guanine nucleotide binding protein |
| | | | | (G protein), alpha z polypeptide (GNAZ), mRNA |
| | (SC 7) | M | PRKAR2B | Homo sapiens protein kinase, cAMP-dependent, |
| | | | | regulatory, type II, beta (PRKAR2B), mRNA |
| | (SC 7) | M | LTBP1 | Homo sapiens latent transforming growth factor |
| | | | | beta binding protein 1 (LTBP1), transcript variant |
| | | | | 1, mRNA |
| | (SC 7) | M | CLEC2 | Homo sapiens C-type lectin-like receptor-2 |
| | | | | (CLEC2), mRNA |
| | (SC 7) | M | TAL1 | Homo sapiens T-cell acute lymphocytic leukemia 1 |
| | | | | (TAL1), mRNA |
| | (SC 7) | M | PTK2 | Homo sapiens PTK2 protein tyrosine kinase 2 |
| | | | | (PTK2), transcript variant 2, mRNA |
| | (SC 7) | M | SDPR | Homo sapiens serum deprivation response |
| | | | | (phosphatidylserine binding protein) (SDPR), |
| | | | | mRNA |
| | (SC 7) | M | PROS1 | Homo sapiens protein S (alpha) (PROS1), mRNA |
| | (SC 7) | M | RUFY1 | Homo sapiens RUN and FYVE domain containing |
| | | | | 1 (RUFY1), mRNA |
| | (SC 7) | M | SLC24A3 | Homo sapiens solute carrier family 24 |
| | | | | (sodium/potassium/calcium exchanger), member 3 |
| | | | | (SLC24A3), mRNA |
| | (SC 7) | M | MAOB | Homo sapiens monoamine oxidase B (MAOB), |
| | | | | nuclear gene encoding mitochondrial protein, |
| | | | | mRNA |
| | (SC 7) | M | ESAM | Homo sapiens endothelial cell adhesion molecule |
| | | | | (ESAM), mRNA |
| | (SC 7) | M | WASF3 | Homo sapiens WAS protein family, member 3 |
| | | | | (WASF3), mRNA |
| | (SC 7) | M | SH3BGRL2 | Homo sapiens SH3 domain binding glutamic acid- |
| | | | | rich protein like 2 (SH3BGRL2), mRNA |
| | (SC 7) | M | TUBB1 | Homo sapiens tubulin, beta 1 (TUBB1), mRNA |
| | (SC 7) | M | CTDSPL | Homo sapiens CTD (carboxy-terminal domain, |
| | | | | RNA polymerase II, polypeptide A) small |
| | | | | phosphatase-like (CTDSPL), mRNA |
| | (SC 7) | M | CML2 | Homo sapiens putative N-acetyltransferase |
| | | | | Camello 2 (CML2), mRNA |
| | (SC 7) | M | F11R | Homo sapiens F11 receptor (F11R), transcript |
| | | | | variant 5, mRNA |
| | (SC 7) | M | N/A | A_24_P333372Homo sapiens cDNA FLJ35984 |
| | | | | fis, clone TESTI2014097, highly similar to |
| | | | | V_segment translation product |
| | (SC 7) | M | PTPRF | Homo sapiens protein tyrosine phosphatase, |
| | | | | receptor type, F (PTPRF), transcript variant 2, |
| | | | | mRNA |
| | (SC 7) | M | N/A | Human DNA sequence from clone RP3-370M22 |
| | | | | on chromosome 22, complete sequence. |
| | (SC 7) | M | MFAP3L | Homo sapiens microfibrillar-associated protein 3- |
| | | | | like (MFAP3L), mRNA |
| | (SC 7) | M | PRKAR2B | Homo sapiens protein kinase, cAMP-dependent, |
| | | | | regulatory, type II, beta (PRKAR2B), mRNA |
| | (SC 7) | M | N/A | A_32_P196142 AC090409Homo sapiens |
| | | | | chromosome 18, clone RP11-879F14, complete |
| | | | | sequence. |
| | (SC 7) | M | TPM1 | Homo sapiens tropomyosin 1 (alpha) (TPM1), |
| | | | | transcript variant 3, mRNA [NM_001018004] |
| | SC 7 | P | GATA1 | GATA binding protein 1 (globin transcription factor |
| | | | | 1) |
| | SC 7 | P | EPOR | erythropoietin receptor |
| | SC 7 | B | ITGA2B | Homo sapiens integrin, alpha 2b (platelet |
| | | | | glycoprotein IIb of IIb/IIIa complex, antigen |
| | | | | CD41B) (ITGA2B), mRNA |
| | SC 7 | B | C6orf25 | Homo sapiens chromosome 6 open reading frame |
| | | | | 25 (C6orf25), transcript variant 6, mRNA |
| | SC 7 | B | C6orf25 | Homo sapiens chromosome 6 open reading frame |
| | | | | 25 (C6orf25), transcript variant 7, mRNA |
| | SC 7 | B | MPL | Homo sapiens myeloproliferative leukemia virus |
| | | | | oncogene (MPL), mRNA |
| | SC 7 | B | INPP5A | Homo sapiens inositol polyphosphate-5- |
| | | | | phosphatase, 40 kDa (INPP5A), mRNA |
| | SC 7 | B | TNFSF4 | Homo sapiens tumor necrosis factor (ligand) |
| | | | | superfamily, member 4 (tax-transcriptionally |
| | | | | activated glycoprotein 1, 34 kDa) (TNFSF4), |
| | | | | mRNA |
| | SC 7 | B | SELP | Homo sapiens selectin P (granule membrane |
| | | | | protein 140 kDa, antigen CD62) (SELP), mRNA |
| | SC 7 | B | PF4 | Homo sapiens platelet factor 4 (chemokine |
| | | | | (C—X—C motif) ligand 4) (PF4), mRNA |

TABLE 8
the T/B Cell Regulation Cluster

| correlation | | | gene | |
|---|---|---|---|---|
| coefficient | subcluster | source | symbol | annotation |
| 0.48 | (SC 8) | M | WNT10A | Homo sapiens wingless-type MMTV integration site |
| | | | | family, member 10A (WNT10A), mRNA |
| | (SC 8) | M | NOSIP | Homo sapiens nitric oxide synthase interacting |
| | | | | protein (NOSIP), mRNA |
| | (SC 8) | M | CRIP2 | Homo sapiens cysteine-rich protein 2 (CRIP2), |
| | | | | mRNA |
| | (SC 8) | M | ASNS | Homo sapiens asparagine synthetase (ASNS), |
| | | | | transcript variant 3, mRNA |
| | (SC 8) | M | ZNF395 | Homo sapiens zinc finger protein 395 (ZNF395), |
| | | | | mRNA |
| | (SC 8) | M | TLE2 | Homo sapiens transducin-like enhancer of split 2 |
| | | | | (E(sp1) homolog, Drosophila) (TLE2), mRNA |
| | (SC 8) | M | EPHA1 | Homo sapiens EphA1 (EPHA1), mRNA |
| | (SC 8) | M | C6orf60 | Homo sapiens chromosome 6 open reading frame |
| | | | | 60 (C6orf60), mRNA |
| | (SC 8) | M | CTSF | Homo sapiens cathepsin F (CTSF), mRNA |
| | (SC 8) | M | CAMK4 | Homo sapiens calcium/calmodulin-dependent |
| | | | | protein kinase IV (CAMK4), mRNA |
| | (SC 8) | M | EBI2 | Homo sapiens Epstein-Barr virus induced gene 2 |
| | | | | (lymphocyte-specific G protein-coupled receptor) |
| | | | | (EBI2), mRNA |
| | (SC 8) | M | LOC129293 | PREDICTED: Homo sapiens hypothetical protein |
| | | | | LOC129293 (LOC129293), mRNA |
| | (SC 8) | M | N/A | Agilent Probe (A_23_P308924) AC007619 Homo |
| | | | | sapiens 12 BAC RP11-253l19 (Roswell Park |
| | | | | Cancer Institute Human BAC Library) complete |
| | | | | sequence. |
| | (SC 8) | M | TCEA3 | Homo sapiens transcription elongation factor A |
| | | | | (SII), 3 (TCEA3), mRNA |
| | (SC 8) | M | TCEA3 | Homo sapiens transcription elongation factor A |
| | | | | (SII), 3 (TCEA3), mRNA |
| | (SC 8) | M | N/A | Agilent Probe (A_23_P353905) AC007619 Homo |
| | | | | sapiens 12 BAC RP11-253l19 (Roswell Park |
| | | | | Cancer Institute Human BAC Library) complete |
| | | | | sequence. |
| | (SC 8) | M | ITK | Homo sapiens IL2-inducible T-cell kinase (ITK), |
| | | | | mRNA |
| | (SC 8) | M | PLXDC1 | Homo sapiens plexin domain containing 1 |
| | | | | (PLXDC1), mRNA |
| | (SC 8) | M | KIAA1407 | Homo sapiens KIAA1407 protein (KIAA1407), |
| | | | | mRNA |
| | (SC 8) | M | GRAP | GRB2-related adaptor protein |
| | (SC 8) | M | GRAP | Homo sapiens chromosome 17, clone RP11- |
| | | | | 160E2, complete sequence. |
| | (SC 8) | M | LOC129293 | Homo sapiens hypothetical protein LOC129293, |
| | | | | mRNA (cDNA clone IMAGE: 5762496), partial cds. |
| | | | | [BC051789] |
| | (SC 8) | M | RHOH | Homo sapiens ras homolog gene family, member H |
| | | | | (RHOH), mRNA |
| | (SC 8) | M | GCN5L2 | Homo sapiens GCN5 general control of amino-acid |
| | | | | synthesis 5-like 2 (yeast) (GCN5L2), mRNA |
| | (SC 8) | M | TCF7 | Homo sapiens transcription factor 7 (T-cell specific, |
| | | | | HMG-box) (TCF7), transcript variant 5, mRNA |
| | (SC 8) | M | IL23A | Homo sapiens interleukin 23, alpha subunit p19 |
| | | | | (IL23A), mRNA |
| | (SC 8) | M | GPRASP1 | Homo sapiens G protein-coupled receptor- |
| | | | | associated sorting protein (GASP), mRNA |
| | (SC 8) | M | ZNF395 | Homo sapiens zinc finger protein 395 (ZNF395). |
| | | | | mRNA |
| | (SC 8) | M | NOSIP | Homo sapiens nitric oxide synthase interacting |
| | | | | protein (NOSIP), mRNA |
| | (SC 8) | M | LTBP3 | Homo sapiens latent transforming growth factor |
| | | | | beta binding protein 3 (LTBP3), mRNA |
| | (SC 8) | M | TCEA3 | Homo sapiens transcription elongation factor A |
| | | | | (SII), 3 (TCEA3), mRNA |
| | (SC 8) | M | CD5 | Homo sapiens CD5 antigen (p56-62) (CD5), mRNA |
| | (SC 8) | M | LOC401905 | PREDICTED: Homo sapiens similar to zinc finger |
| | | | | protein 91 (HPF7, HTF10) (LOC401905), mRNA |
| | (SC 8) | M | AP3M2 | Homo sapiens adaptor-related protein complex 3, |
| | | | | mu 2 subunit (AP3M2), mRNA |
| | (SC 8) | M | TMEM16J | Homo sapiens transmembrane protein 16J |
| | | | | (TMEM16J), mRNA. |
| | (SC 8) | M | N/A | Agilent Probe (A_32_P203728 AC012020 Homo |
| | | | | sapiens 3 BAC RP11-861A13 (Roswell Park |
| | | | | Cancer Institute Human BAC Library) complete |
| | | | | sequence. |
| | (SC 8) | M | N/A | Agilent Probe (A_32_P231493) Homo sapiens |
| | | | | clone IMAGE: 1257951, mRNA sequence |
| | (SC 8) | M | N/A | Agilent Probe (A_32_P874898) U66059 Human |
| | | | | germline T-cell receptor beta chain Dopamine-beta- |
| | | | | hydroxylase-like, TRY1, TRY2, TRY3 |
| | | | | TCRBV27S1P, TCRBV22S1A2N1T, |
| | | | | TCRBV9S1A1T, TCRBV7S1A1N2T, |
| | | | | TCRBV5S1A1T, TCRBV13S3, TCRBV6S7P, |
| | | | | TCRBV7S3A2T, TCRBV13S2A1T, |
| | | | | TCRBV9S2A2PT, TCRBV7S2A1N4T, |
| | | | | TCRBV13S9/13S2A1T, TCRBV6S5A1N1, |
| | | | | TCRBV30S1P, TCRBV31S1, TCRBV13S5, |
| | | | | TCRBV6S1A1N1, TCRBV32S1P, TCRBV5S5P, |
| | | | | TCRBV1S1A1N1, TCRBV12S2A1T, TCRBV21S1, |
| | | | | TCRBV8S4P, TCRBV12S3, TCRBV21S3A2N2T, |
| | | | | TCRBV8S5P, TCRBV13S1 genes from bases 1 to |
| | | | | 267156 (section 1 of 3). |
| | SC 8 | P | CCR7 | chemokine (C—C motif) receptor 7 |
| | SC 8 | P | HZF12 | zinc finger protein 101 |
| | SC 8 | P | IL2RA | interleukin 2 receptor, alpha |
| | SC 8 | P | LEF1 | lymphoid enhancer-binding factor 1 |
| | SC 8 | P | MYC | nucleolar protein 3 (apoptosis repressor withCARD |
| | | | | domain) |
| | SC 8 | P | TNFSF5 | tumor necrosis factor (ligand) superfamily, member |
| | | | | 5 (hyper-IgM syndrome) |
| | SC 8 | B | IL7R | Homo sapiens interleukin 7 receptor (IL7R), mRNA |
| | SC 8 | B | TNFRSF7 | Homo sapiens tumor necrosis factor receptor |
| | | | | superfamily, member 7 (TNFRSF7), mRNA |
| | SC 8 | B | FLT3LG | Homo sapiens fims-related tyrosine kinase 3 ligand |
| | | | | (FLT3LG), mRNA |
| | SC 8 | B | CD28 | Homo sapiens CD28 antigen (Tp44) (CD28), mRNA |

TABLE 9
the Transcription Control Cluster

| correlation | | | gene | |
|---|---|---|---|---|
| coefficient | subcluster | source | symbol | annotation |
| 0.33 | SC 9 | P | NCBP2 | nuclear cap binding protein |
| | | | | subunit 2, 20 kDa |
| | SC 9 | P | DATF1 | Death associated |
| | | | | Transcription factor |
| | SC 9 | P | TERF2 | telomeric repeat binding |
| | | | | factor 2 |
| | SC 9 | P | POT1 | protection of telomeres 1 |

TABLE 10
the T Cell Cluster

| correlation | | | gene | |
|---|---|---|---|---|
| coefficient | subcluster | source | symbol | annotation |
| 0.65 | (SC 10) | M | GZMM | Homo sapiens granzyme M (lymphocyte met-ase 1) |
| | | | | (GZMM), mRNA |
| | (SC 10) | M | GZMA | Homo sapiens granzyme A (granzyme 1, cytotoxic T- |
| | | | | lymphocyte-associated serine esterase 3) (GZMA), |
| | | | | mRNA |
| | (SC 10) | M | MARLIN1 | Homo sapiens multiple coiled-coil GABABR1-binding |
| | | | | protein (MARLIN1), mRNA |
| | (SC 10) | M | PPP2R2B | Homo sapiens protein phosphatase 2 (formerly 2A), |
| | | | | regulatory subunit B (PR 52), beta isoform |
| | | | | (PPP2R2B), transcript variant 1, mRNA |
| | (SC 10) | M | MCOLN2 | Homo sapiens mucolipin 2 (MCOLN2), mRNA |
| | (SC 10) | M | RASGEF1A | Homo sapiens RasGEF domain family, member 1A |
| | | | | (RASGEF1A), mRNA |
| | (SC 10) | M | SH2D1A | Homo sapiens SH2 domain protein 1A, Duncan's |
| | | | | disease (lymphoproliferative syndrome) (SH2D1A), |
| | | | | mRNA |
| | (SC 10) | M | FLJ39873 | Homo sapiens hypothetical protein FLJ39873 |
| | | | | (FLJ39873), mRNA |
| | (SC 10) | M | IFIX | Homo sapiens interferon-inducible protein X (IFIX), |
| | | | | transcript variant a2, mRNA |
| | (SC 10) | M | KLRK1 | Homo sapiens killer cell lectin-like receptor subfamily |
| | | | | K, member 1 (KLRK1), mRNA |
| | (SC 10) | M | N/A | Agilent probe (A_24_P911973) Accession |
| | | | | NM_139211 Homo sapiens homeodomain-only |
| | | | | protein (HOP), transcript variant 2, mRNA |
| | (SC 10) | M | N/A | Agilent probe (A_24_P945283) Homo sapiens mRNA |
| | | | | for KIAA1232 protein, partial cds |
| | (SC 10) | P | CCL5 | chemokine (C—C motif) ligand 5 |
| | SC 10 | P | PRDM1 | PR domain containing 1, with ZNF domain |
| | SC 10 | P | RUNX3 | runt-related transcription factor 3 |
| | SC 10 | P | TAP1 | transporter 1, ATP-binding cassette, sub-family B |
| | | | | (MDR/TAP) |
| | SC 10 | P | IFNG | interferon, gamma |
| | SC 10 | B | LAG3 | Homo sapiens lymphocyte-activation gene 3 (LAG3), |
| | | | | mRNA |
| | SC 10 | B | PDCD1 | Homo sapiens programmed cell death 1 (PDCD1), |
| | | | | mRNA |
| | SC 10 | B | CD8B1 | Homo sapiens CD8 antigen, beta polypeptide 1 (p37) |
| | | | | (CD8B1), transcript variant 1, mRNA |
| | SC 1 | B | ADA | Homo sapiens adenosine deaminase (ADA), mRNA |
| | SC 10 | B | CD160 | Homo sapiens CD160 antigen (CD160), mRNA |
| | SC 10 | B | CD8B1 | Homo sapiens CD8 antigen, beta polypeptide 1 (p37) |
| | | | | (CD8B1), transcript variant 2, mRNA |
| | SC 10 | B | CD8A | Homo sapiens CD8 antigen, alpha polypeptide (p32) |
| | | | | (CD8A), transcript variant 2, mRNA |

TABLE 11
the Inflammatory Cell Recruitment Cluster

| correlation | | | gene | |
|---|---|---|---|---|
| coefficient | subcluster | source | symbol | annotation |
| 0.62 | (SC 11) | M | SCAP1 | Homo sapiens src family associated phosphoprotein |
| | | | | 1 (SCAP1), mRNA |
| | (SC 11) | M | MGC45416 | Homo sapiens hypothetical protein MGC45416 |
| | | | | (MGC45416), mRNA |
| | (SC 11) | M | RASGRP1 | Homo sapiens RAS guanyl releasing protein 1 |
| | | | | (calcium and DAG-regulated) (RASGRP1), mRNA |
| | (SC 11) | M | C6orf129 | Homo sapiens chromosome 6 open reading frame |
| | | | | 129 (C6orf129), mRNA |
| | (SC 11) | M | BIN1 | Homo sapiens bridging integrator 1 (BIN1), transcript |
| | | | | variant 10, mRNA |
| | (SC 11) | M | PCBP4 | Homo sapiens poly(rC) binding protein 4 (PCBP4), |
| | | | | transcript variant 4, mRNA |
| | (SC 11) | M | DYRK2 | Homo sapiens dual-specificity tyrosine-(Y)- |
| | | | | phosphorylation regulated kinase 2 (DYRK2), |
| | | | | transcript variant 2, mRNA |
| | (SC 11) | M | CD6 | Homo sapiens T cell surface glycoprotein CD6 |
| | | | | isoform (CD6) gene, exons 2-13, and complete cds, |
| | | | | alternatively spliced. |
| | (SC 11) | M | SIAT8A | Homo sapiens sialyltransferase 8A (alpha-N- |
| | | | | acetylneuraminate: alpha-2,8-sialyltransferase, GD3 |
| | | | | synthase) (SIAT8A), mRNA |
| | (SC 11) | M | ZAP70 | Homo sapiens zeta-chain (TCR) associated protein |
| | | | | kinase 70 kDa (ZAP70), transcript variant 2, mRNA |
| | (SC 11) | M | LOC389289 | PREDICTED: Homo sapiens similar to annexin II |
| | | | | receptor (LOC389289), mRNA |
| | (SC 11) | M | LAT | Homo sapiens linker for activation of T cells (LAT), |
| | | | | mRNA |
| | (SC 11) | M | CD96 | Homo sapiens CD96 antigen (CD96), transcript |
| | | | | variant 1, mRNA |
| | (SC 11) | M | SLC2A1 | Homo sapiens solute carrier family 2 (facilitated |
| | | | | glucose transporter), member 1 (SLC2A1), mRNA |
| | (SC 11) | M | MGC10992 | Homo sapiens hypothetical protein LOC92922 |
| | | | | (MGC10992), mRNA |
| | (SC 11) | M | FLJ12953 | Homo sapiens hypothetical protein FLJ12953 similar |
| | | | | to Mus musculus D3Mm3e (FLJ12953), mRNA |
| | (SC 11) | M | FLJ21438 | PREDICTED: Homo sapiens hypothetical protein |
| | | | | FLJ21438 (FLJ21438), mRNA |
| | (SC 11) | M | STAG3 | Homo sapiens stromal antigen 3 (STAG3), mRNA |
| | (SC 11) | M | SIGIRR | Homo sapiens single Ig IL-1R-related molecule |
| | | | | (SIGIRR), mRNA |
| | (SC 11) | M | 0 | Homo sapiens cDNA FLJ14201 fis, clone |
| | | | | NT2RP3002955 |
| | SC 11 | P | FCRH3 | Fc receptor-like protein 3 |
| | SC 11 | P | TRAC | T cell receptor alpha constant |
| | SC 11 | P | TRBC1 | T cell receptor beta constant 1 |
| | SC 11 | B | LCK | Homo sapiens lymphocyte-specific protein tyrosine |
| | | | | kinase (LCK), mRNA |
| | SC 11 | B | CXCR3 | Homo sapiens chemokine (C—X—C motif) receptor 3 |
| | | | | (CXCR3), mRNA |
| | SC 11 | B | GATA3 | Homo sapiens GATA binding protein 3 (GATA3), |
| | | | | mRNA |
| | SC 11 | B | ITGB7 | Homo sapiens integrin, beta 7 (ITGB7), mRNA |
| | SC 11 | B | PRKCQ | Homo sapiens protein kinase C, theta (PRKCQ), |
| | | | | mRNA |

TABLE 12
the Transcription Factor Related Cluster

| correlation | | | gene | |
|---|---|---|---|---|
| coefficient | subcluster | source | symbol | annotation |
| 0.64 | SC 12 | P | RUNX1 | runt-related transcription factor 1 (acute myeloid |
| | | | | eukemia 1; aml1 oncogene) |
| | SC 13 | B | ZFYVE27 | Homo sapiens zinc finger, FYVE domain containing |
| | | | | 27 (ZFYVE27), mRNA |
| | SC 12 | B | KPNA6 | Homo sapiens karyopherin alpha 6 (importin alpha |
| | | | | 7) (KPNA6), mRNA |
| | SC 9 | B | KPNB1 | Homo sapiens karyopherin (importin) beta 1 |
| | | | | (KPNB1), mRNA |
| | (SC 12) | M | CYHR1 | Homo sapiens cysteine and histidine rich 1 |
| | | | | (CYHR1), mRNA |
| | (SC 12) | M | SNAPC4 | Homo sapiens small nuclear RNA activating |
| | | | | complex, polypeptide 4, 190 kDa (SNAPC4), mRNA |
| | (SC 12) | M | ARFGAP1 | Homo sapiens ADP-ribosylation factor GTPase |
| | | | | activating protein 1 (ARFGAP1), transcript variant |
| | | | | 2, mRNA |
| | (SC 12) | M | ZNF278 | Homo sapiens zinc finger protein 278 (ZNF278), |
| | | | | transcript variant 3. mRNA |
| | (SC 12) | M | ARHGAP1 | Homo sapiens Rho GTPase activating protein 1 |
| | | | | (ARHGAP1), mRNA |
| | (SC 12) | M | ZCWCC3 | Homo sapiens zinc finger, CW-type with coiled-coil |
| | | | | domain 3 (ZCWCC3), mRNA |
| | (SC 12) | M | RBBP4 | Homo sapiens retinoblastoma binding protein 4 |
| | | | | (RBBP4), mRNA |
| | (SC 12) | M | MGRN1 | Homo sapiens mahogunin, ring finger 1 (MGRN1), |
| | | | | mRNA |
| | (SC 12) | M | COL4A3BP | Homo sapiens collagen, type IV, alpha 3 |
| | | | | (Goodpasture antigen) binding protein |
| | | | | (COL4A3BP), transcript variant 1, mRNA |
| | (SC 12) | M | MYBBP1A | Homo sapiens MYB binding protein (P160) 1a |
| | | | | (MYBBP1A), mRNA |
| | (SC 12) | M | FAM31C | Homo sapiens family with sequence similarity 31, |
| | | | | member C (FAM31C), mRNA |
| | (SC 12) | M | LOC283874 | Homo sapiens hypothetical protein LOC283874 |
| | | | | (LOC283874), mRNA [NM_001012731] |
| | (SC 12) | M | GOSR1 | Homo sapiens golgi SNAP receptor complex |
| | | | | member 1 (GOSR1), mRNA |
| | (SC 12) | M | PITPN | Homo sapiens phosphotidylinositol transfer protein |
| | | | | (PITPN), mRNA |
| | (SC 12) | M | KIAA0261 | Homo sapiens KIAA0261 (KIAA0261), mRNA |
| | (SC 12) | M | ZNF625 | Homo sapiens zinc finger protein 625 (ZNF625), |
| | | | | mRNA |
| | (SC 12) | M | EDEM1 | Homo sapiens ER degradation enhancer, |
| | | | | mannosidase alpha-like 1 (EDEM1), mRNA |
| | (SC 12) | M | RAB43 | member RAS oncogene family |

TABLE 13
the Dendritic Cell Maturation Cluster

| correlation | | | gene | |
|---|---|---|---|---|
| coefficient | subcluster | source | symbol | annotation |
| 0.50 | (SC 13) | M | CTDP1 | Homo sapiens CTD (carboxy-terminal domain, RNA |
| | | | | polymerase II, polypeptide A) phosphatase, subunit 1 |
| | | | | (CTDP1), transcript variant FCP1b, mRNA |
| | SC 13 | P | LPPR2 | lipid phosphate phosphatase-related protein type 2 |
| | SC 13 | P | TBXAS1 | thromboxane A synthase 1 |
| | SC 12 | B | VAV1 | Homo sapiens vav 1 oncogene (VAV1), mRNA |
| | SC 13 | B | NOTCH1 | Homo sapiens Notch homolog 1, translocation- |
| | | | | associated (Drosophila) (NOTCH1), mRNA |
| | SC 3 | B | SIRPB1 | Homo sapiens signal-regulatory protein beta 1 |
| | | | | (SIRPB1), mRNA |
| | (SC 13) | M | ICMT | Homo sapiens isoprenylcysteine carboxyl |
| | | | | methyltransferase (ICMT), transcript variant 2, mRNA |
| | (SC 13) | M | GALNS | Homo sapiens galactosamine (N-acetyl)-6-sulfate |
| | | | | sulfatase (Morquio syndrome, mucopolysaccharidosis |
| | | | | type IVA) (GALNS), mRNA |
| | (SC 13) | M | FUS | Homo sapiens fusion (involved in t(12; 16) in malignant |
| | | | | liposarcoma) (FUS), mRNA |
| | (SC 13) | M | EIF2B5 | Homo sapiens eukaryotic translation initiation factor |
| | | | | 2B, subunit 5 epsilon, 82 kDa (EIF2B5), mRNA |
| | (SC 13) | M | CGI-41 | Homo sapiens CGI-41 protein (CGI-41), mRNA |
| | (SC 13) | M | TUBGCP2 | Homo sapiens tubulin, gamma complex associated |
| | | | | protein 2 (TUBGCP2), mRNA |
| | (SC 13) | M | HEM1 | Homo sapiens hematopoietic protein 1 (HEM1), mRNA |
| | (SC 13) | M | FBXO18 | Homo sapiens F-box protein, helicase, 18 (FBXO18), |
| | | | | transcript variant 2, mRNA |
| | (SC 13) | M | DUSP3 | Homo sapiens dual specificity phosphatase 3 (vaccinia |
| | | | | virus phosphatase VH1-related) (DUSP3), mRNA |
| | (SC 13) | M | LOC92799 | Homo sapiens hypothetical protein BC007653 |
| | | | | (LOC92799), mRNA |
| | (SC 13) | M | BTK | Homo sapiens Bruton agammaglobulinemia tyrosine |
| | | | | kinase (BTK), mRNA |
| | (SC 13) | M | PD2 | Homo sapiens hypothetical protein F23149_1 (PD2), |
| | | | | mRNA |
| | (SC 13) | M | PANK4 | Homo sapiens pantothenate kinase 4 (PANK4), mRNA |
| | (SC 13) | M | RHOG | Homo sapiens ras homolog gene family, member G |
| | | | | (rho G) (RHOG), mRNA |
| | (SC 13) | M | KARS | Homo sapiens lysyl-tRNA synthetase (KARS), mRNA |
| | (SC 13) | M | IFI30 | Homo sapiens interferon, gamma-inducible protein 30 |
| | | | | (IFI30), mRNA |
| | (SC 13) | M | ARRB2 | Homo sapiens arrestin, beta 2 (ARRB2), transcript |
| | | | | variant 2, mRNA |
| | (SC 13) | M | NUMA1 | Homo sapiens nuclear mitotic apparatus protein 1 |
| | | | | (NUMA1), mRNA |
| | (SC 13) | M | SENP3 | Homo sapiens SUMO1/sentrin/SMT3 specific protease |
| | | | | 3 (SENP3), mRNA |
| | (SC 13) | M | DBNL | PREDICTED: Homo sapiens drebrin-like (DBNL), |
| | | | | mRNA |
| | (SC 13) | M | TRIM26 | Homo sapiens tripartite motif-containing 26 (TRIM26), |
| | | | | mRNA |
| | (SC 13) | M | ZBED1 | Homo sapiens zinc finger, BED domain containing 1 |
| | | | | (ZBED1), mRNA |
| | (SC 13) | M | SERPINA1 | Homo sapiens serine (or cysteine) proteinase inhibitor, |
| | | | | clade A (alpha-1 antiproteinase, antitrypsin), member 1 |
| | | | | (SERPINA1), mRNA |
| | (SC 13) | M | TTC15 | Homo sapiens tetratricopeptide repeat domain 15 |
| | | | | (TTC15), mRNA |
| | (SC 13) | M | CDK5RAP1 | Homo sapiens CDK5 regulatory subunit associated |
| | | | | protein 1 (CDK5RAP1), transcript variant 2, mRNA |
| | (SC 13) | M | PLD3 | Homo sapiens phospholipase D3 (PLD3), mRNA |
| | (SC 13) | M | KNS2 | Homo sapiens kinesin 2 60/70 kDa (KNS2), mRNA |
| | (SC 13) | M | C20orf27 | Homo sapiens chromosome 20 open reading frame 27 |
| | | | | (C20orf27), mRNA |
| | (SC 13) | M | GYG | Homo sapiens glycogenin (GYG), mRNA |
| | (SC 13) | M | CRSP8 | Homo sapiens cofactor required for Sp1 transcriptional |
| | | | | activation, subunit 8, 34 kDa (CRSP8), mRNA |
| | (SC 13) | M | ACO2 | Homo sapiens aconitase 2, mitochondrial (ACO2), |
| | | | | nuclear gene encoding mitochondrial protein, mRNA |
| | (SC 13) | M | GRINA | PREDICTED: Homo sapiens glutamate receptor, |
| | | | | ionotropic, N-methyl D-asparate-associated protein 1 |
| | | | | (glutamate binding) (GRINA), mRNA |
| | (SC 13) | M | XPO6 | Homo sapiens exportin 6 (XPO6), mRNA |
| | (SC 13) | M | G6PD | Homo sapiens glucose-6-phosphate dehydrogenase |
| | | | | (G6PD), nuclear gene encoding mitochondrial protein, |
| | | | | mRNA |
| | (SC 13) | M | TRAP1 | Homo sapiens TNF receptor-associated protein 1 |
| | | | | (TRAP1), mRNA |
| | (SC 13) | M | BLCAP | Homo sapiens bladder cancer associated protein |
| | | | | (BLCAP), mRNA |
| | (SC 13) | M | VPS33B | Homo sapiens vacuolar protein sorting 33B (yeast) |
| | | | | (VPS33B), mRNA |
| | (SC 13) | M | CTSD | Homo sapiens cathepsin D (lysosomal aspartyl |
| | | | | protease) (CTSD), mRNA |
| | (SC 13) | M | COX10 | Homo sapiens COX10 homolog, cytochrome c oxidase |
| | | | | assembly protein, heme A: farnesyltransferase (yeast) |
| | | | | (COX10), nuclear gene encoding mitochondrial |
| | | | | protein, mRNA |
| | (SC 13) | M | FLOT2 | Homo sapiens flotillin 2 (FLOT2), mRNA |
| | (SC 13) | M | FLJ12886 | Homo sapiens hypothetical protein FLJ12886 |
| | | | | (FLJ12886), mRNA |
| | (SC 13) | M | AAMP | Homo sapiens angio-associated, migratory cell protein |
| | | | | (AAMP), mRNA |
| | (SC 13) | M | CTNNA1 | Homo sapiens catenin (cadherin-associated protein), |
| | | | | alpha 1, 102 kDa (CTNNA1), mRNA |
| | (SC 13) | M | WARS | Homo sapiens tryptophanyl-tRNA synthetase (WARS), |
| | | | | transcript variant 4, mRNA |
| | (SC 13) | M | CECR1 | Homo sapiens cat eye syndrome chromosome region, |
| | | | | candidate 1 (CECR1), transcript variant 1, mRNA |
| | (SC 13) | M | CD1D | Homo sapiens CD1D antigen, d polypeptide (CD1D), |
| | | | | mRNA |
| | (SC 13) | M | GRB2 | Homo sapiens growth factor receptor-bound protein 2 |
| | | | | (GRB2), transcript variant 2, mRNA |
| | (SC 13) | M | ALDH1A1 | Homo sapiens aldehyde dehydrogenase 1 family, |
| | | | | member A1 (ALDH1A1), mRNA |
| | (SC 13) | M | ALDH3B1 | Homo sapiens aldehyde dehydrogenase 3 family, |
| | | | | member B1 (ALDH3B1), mRNA |
| | (SC 13) | M | N/A | Agilent probe (A_24_P246963) Accession AC055716 |
| | | | | Homo sapiens 12 BAC RP11-641A6 (Roswell Park |
| | | | | Cancer Institute Human BAC Library) complete |
| | | | | sequence. |
| | (SC 13) | M | N/A | Agilent probe (A_24_P306355) Accession AC005815 |
| | | | | Homo sapiens chromosome 22 clone 239d10 map |
| | | | | 22q11, complete sequence. |
| | (SC 13) | M | N/A | Agilent probe (A_24_P451992) Accession AC009831 |
| | | | | Homo sapiens chromosome, clone RP11-326K13, |
| | | | | complete sequence. |
| | (SC 13) | M | N/A | Agilent probe (A_24_P67748) Accession AL512427 |
| | | | | Human DNA sequence from clone RP11-325M4 on |
| | | | | chromosome 6 Contains a retinoblastoma binding |
| | | | | protein 4 (RBBP4) pseudogene, complete sequence. |
| | (SC 13) | M | C2orf18 | Homo sapiens chromosome 2 open reading frame 18, |
| | | | | mRNA (cDNA clone IMAGE: 3860139), complete cds, |
| | | | | [BC016389] |
| | (SC 13) | M | EWSR1 | Homo sapiens Ewing sarcoma breakpoint region 1 |
| | | | | (EWSR1), transcript variant EWS-b, mRNA |

TABLE 14
the Cell Activation Cluster

| correlation | | | gene | |
|---|---|---|---|---|
| coefficient | subcluster | source | symbol | annotation |
| 0.42 | SC 14 | P | CXCL1 | Chemokine (C—X—C |
| | | | | motif) ligand 1 |
| | SC 14 | P | GPI | glucose phosphate |
| | | | | isomerase |
| | SC 14 | P | CLECSF5 | C-type (calcium dependent, |
| | | | | carbohydrate-recognition |
| | | | | domain) lectin, superfamily |
| | | | | member 5 |

TABLE 15
the Cytotoxic T Cell Cluster

| correlation | | | gene | |
|---|---|---|---|---|
| coefficient | subcluster | source | symbol | annotation |
| 0.65 | (SC 15) | M | EDG8 | Homo sapiens endothelial differentiation, sphingolipid |
| | | | | G-protein-coupled receptor, 8 (EDG8), mRNA |
| | (SC 15) | M | EDG8 | Homo sapiens endothelial differentiation, sphingolipid |
| | | | | G-protein-coupled receptor, 8 (EDG8), mRNA |
| | (SC 15) | M | BATF | Homo sapiens basic leucine zipper transcription |
| | | | | factor, ATF-like (BATF), mRNA |
| | (SC 15) | M | CTSW | Homo sapiens cathepsin W (lymphopain) (CTSW), |
| | | | | mRNA |
| | (SC 15) | M | TBX21 | Homo sapiens T-box 21 (TBX21), mRNA |
| | (SC 15) | M | PRSS23 | Homo sapiens protease, serine, 23 (PRSS23), mRNA |
| | (SC 15) | M | PTPN7 | Homo sapiens protein tyrosine phosphatase, non- |
| | | | | receptor type 7 (PTPN7), transcript variant 3, mRNA |
| | (SC 15) | M | PTGDR | Homo sapiens prostaglandin D2 receptor (DP) |
| | | | | (PTGDR), mRNA |
| | (SC 15) | M | CHST12 | Homo sapiens carbohydrate (chondroitin 4) |
| | | | | sulfotransferase 12 (CHST12), mRNA |
| | (SC 15) | M | TNFSF6 | Homo sapiens tumor necrosis factor (ligand) |
| | | | | superfamily, member 6 (TNFSF6), mRNA |
| | (SC 15) | M | TTC16 | Homo sapiens tetratricopeptide repeat domain 16 |
| | | | | (TTC16), mRNA |
| | (SC 15) | M | RAB11FIP5 | Homo sapiens RAB11 family interacting protein 5 |
| | | | | (class I) (RAB11FIP5), mRNA |
| | (SC 15) | M | KLRG1 | Homo sapiens killer cell lectin-like receptor subfamily |
| | | | | G, member 1 (KLRG1), mRNA |
| | (SC 15) | M | PLEKHF1 | Homo sapiens pleckstrin homology domain |
| | | | | containing, family F (with FYVE domain) member 1 |
| | | | | (PLEKHF1), mRNA |
| | (SC 15) | M | PLEKHF1 | Homo sapiens pleckstrin homology domain |
| | | | | containing, family F (with FYVE domain) member 1 |
| | | | | (PLEKHF1), mRNA |
| | (SC 15) | M | N/A | Agilient probe (A_24_P106910) Homo sapiens mRNA |
| | | | | for patched variant protein |
| | (SC 15) | M | CTSW | Homo sapiens cathepsin W (lymphopain) (CTSW), |
| | | | | mRNA |
| | (SC 15) | M | TNFSF6 | Homo sapiens tumor necrosis factor (ligand) |
| | | | | superfamily, member 6 (TNFSF6), mRNA |
| | (SC 15) | M | FLJ21069 | Homo sapiens BAC clone RP11-656O12 from 2, |
| | | | | complete sequence. |
| | (SC 15) | M | N/A | Homo sapiens PAC clone RP5-1099N7 from 1, |
| | | | | complete sequence. |
| | (SC 15) | M | GPR68 | Homo sapiens G protein-coupled receptor 68 |
| | | | | (GPR68), mRNA |
| | (SC 15) | M | C9orf81 | Human DNA sequence from clone RP11-336N8 on |
| | | | | chromosome 9q21.11-21.31 Contains a synaptogyrin |
| | | | | 2 (SYNGR2) pseudogene, an argininosuccinate |
| | | | | synthetase (ASS) pseudogene, a ribosomal protein |
| | | | | L21 (RPL21) pseudogene, a CDC28 protein kinase |
| | | | | regulatory subunit 2 (CKS2) pseudogene, the C9orf81 |
| | | | | gene for chromosome 9 open reading frame 81 and a |
| | | | | CpG island, complete sequence. |
| | SC 15 | P | KLRF1 | Killer cell lectin-like receptor subfamily F |
| | SC 15 | P | KLRC1 | killer cell lectin-like receptor subfamily C, member 1 |
| | SC 15 | B | GZMB | Homo sapiens granzyme B (granzyme 2, cytotoxic T- |
| | | | | lymphocyte-associated serine esterase 1) (GZMB), |
| | | | | mRNA |
| | SC 15 | B | PRF1 | Homo sapiens perforin 1 (pore forming protein) |
| | | | | (PRF1), mRNA |
| | SC 15 | B | GNLY | Homo sapiens granulysin (GNLY), transcript variant |
| | | | | 519, mRNA |
| | SC 10 | B | CCL4 | Homo sapiens chemokine (C—C motif) ligand 4 |
| | | | | (CCL4), mRNA |
| | SC 11 | B | CBLB | Homo sapiens Cas-Br-M (murine) ecotropic retroviral |
| | | | | transforming sequence b (CBLB), mRNA |

TABLE 16
the Bone Marrow Stromal Cell Migration Cluster

| correlation | | | gene | |
|---|---|---|---|---|
| coefficient | subcluster | source | symbol | annotation |
| 0.35 | SC 16 | P | CCL3 | Chemokine (C—C motif) |
| | | | | ligand 3 |
| | SC 16 | P | IL8 | interleukin 8 |

TABLE 23
Clinical Characteristics of Study Patients and Samples

| | | | Microarray Study | | | PCR Study: Training | | | PCR Study: Validation | | | |
|---|---|---|---|---|---|---|---|---|---|---|---|---|
| | | | | No | | | No | | High- | Mild | No | |
| | | CARGO | Rejection | Rejection | | Rejection | Rejection | | grade | Rejection | Rejection | |
| | | (N = 629 | (N = 28 | (N = 94 | | (N = 28 | (N = 86 | | Rejection | (N = 69 | (N = 83 | |
| | | patients, | patients, | patients, | | patients, | patients, | | (N = 50 | patients, | patients, | |
| | UNOS | 4917 | 38 | 247 | P | 36 | 109 | P | patients, 62 | 86 | 122 | P |
| | 2003 | samples) | samples) | samples) | value | samples) | samples) | value | samples) | samples) | samples) | value |
| Recipient Age | | | | | | | | | | | | |
| Under 18 | 14.0% | 6.4% | 0.0% | 0.0% | NS | 0.0% | 1.8% | NS | 3.2% | 2.3% | 1.6% | NS |
| 18-34 | 9.4% | 9.9% | 15.8% | 13.0% | | 13.9% | 5.5% | | 14.5% | 15.1% | 13.9% | |
| 35-49 | 21.1% | 17.7% | 23.7% | 12.6% | | 13.9% | 21.1% | | 17.7% | 16.3% | 18.0% | |
| 50-64 | 47.2% | 53.1% | 57.9% | 65.6% | | 69.4% | 56.9% | | 53.2% | 53.5% | 54.9% | |
| 65+ | 8.5% | 12.9% | 2.6% | 8.9% | | 2.8% | 14.7% | | 11.3% | 12.8% | 11.5% | |
| Recipient Race | | | | | | | | | | | | |
| White | 71.1% | 72.3% | 73.7% | 74.1% | NS | 72.2% | 78.9% | NS | 67.7% | 68.6% | 66.4% | NS |
| Black | 16.0% | 17.3% | 21.1% | 15.4% | | 19.4% | 10.1% | | 24.2% | 23.3% | 18.9% | |
| Hispanic | 8.8% | 6.2% | 5.3% | 7.7% | | 8.3% | 6.4% | | 6.5% | 5.8% | 9.0% | |
| Asian | 1.9% | 1.2% | 0.0% | 0.0% | | 0.0% | 1.8% | | 0.0% | 1.2% | 2.5% | |
| Other | 2.1% | 3.1% | 0.0% | 2.8% | | 0.0% | 2.8% | | 1.6% | 1.2% | 3.3% | |
| Recipient Sex | | | | | | | | | | | | |
| Male | 73.6% | 74.6% | 92.1% | 77.3% | NS | 86.1% | 73.4% | NS | 80.6% | 82.6% | 81.1% | NS |
| Female | 26.4% | 25.4% | 7.9% | 22.7% | | 13.9% | 26.6% | | 19.4% | 17.4% | 18.9% | |
| Donor Age | | | | | | | | | | | | |
| Under 18 | 21.6% | 17.9% | 18.4% | 11.4% | NS | 17.1% | 17.5% | NS | 20.0% | 13.3% | 17.1% | NS |
| 18-34 | 44.2% | 46.5% | 47.4% | 49.4% | | 51.4% | 46.6% | | 38.2% | 50.7% | 51.3% | |
| 35-49 | 25.7% | 24.9% | 26.3% | 28.2% | | 22.9% | 24.3% | | 32.7% | 30.7% | 19.7% | |
| 50-64 | 8.2% | 10.5% | 7.9% | 11.0% | | 8.6% | 11.7% | | 9.1% | 5.3% | 11.1% | |
| 65+ | 0.2% | 0.2% | 0.0% | 0.0% | | 0.0% | 0.0% | | 0.0% | 0.0% | 0.9% | |
| Donor Race | | | | | | | | | | | | |
| White | 69.6% | 71.4% | 73.7% | 66.9% | NS | 75.0% | 62.4% | NS | 83.9% | 67.4% | 66.4% | NS |
| Black | 12.0% | 14.2% | 5.3% | 14.6% | | 5.6% | 12.8% | | 8.1% | 11.6% | 13.9% | |
| Hispanic | 15.7% | 11.4% | 21.1% | 16.3% | | 19.4% | 18.3% | | 8.1% | 17.4% | 13.1% | |
| Asian | 1.6% | 0.7% | 0.0% | 2.1% | | 0.0% | 0.9% | | 0.0% | 2.3% | 0.8% | |
| Other | 1.2% | 2.3% | 0.0% | 0.0% | | 0.0% | 5.5% | | 0.0% | 1.2% | 5.7% | |
| Donor Sex | | | | | | | | | | | | |
| Male | 68.4% | 63.7% | 71.1% | 66.1% | NS | 61.1% | 68.5% | NS | 58.1% | 62.8% | 74.2% | NS |
| Female | 31.6% | 36.3% | 28.9% | 33.9% | | 38.9% | 31.5% | | 41.9% | 37.2% | 25.8% | |
| Primary Diagnosis | | | | | | | | | | | | |
| Coronary Artery | 42.1% | 23.8% | 15.8% | 21.9% | NS | 22.2% | 32.1% | NS | 16.1% | 14.0% | 30.3% | 0.025 |
| Disease | | | | | | | | | | | | |
| Cardiomyopathy | 47.0% | 70.0% | 81.6% | 69.2% | | 77.8% | 58.7% | | 79.0% | 72.1% | 64.8% | |
| Congenital Heart | 8.5% | 2.3% | 0.0% | 0.4% | | 0.0% | 1.8% | | 0.0% | 1.2% | 2.5% | |
| Disease | | | | | | | | | | | | |
| Retransplant | 3.3% | 0.5% | 2.6% | 5.3% | | 0.0% | 1.8% | | 3.2% | 7.0% | 0.8% | |
| Valvular Disease | 1.9% | 2.4% | 0.0% | 1.2% | | 0.0% | 1.8% | | 0.0% | 3.5% | 0.8% | |
| Other | 0.5% | 1.0% | 0.0% | 2.0% | | 0.0% | 3.7% | | 1.6% | 2.3% | 0.8% | |
| Immuno- | | | | | | | | | | | | |
| suppression* | | | | | | | | | | | | |
| Cyclosporine | 64.9% | 50.4% | 71.1% | 71.7% | NS | 52.8% | 44.0% | NS | 72.6% | 61.6% | 53.3% | NS |
| FK-506 | 42.9% | 36.6% | 26.3% | 25.5% | | 47.2% | 54.1% | | 25.8% | 37.2% | 38.5% | |
| Mycophenolate | 80.5% | 72.0% | 81.6% | 87.4% | | 72.2% | 78.0% | | 80.6% | 86.0% | 83.6% | |
| Rapamycin | 7.5% | 9.8% | 5.3% | 2.4% | | 22.2% | 14.7% | | 12.9% | 8.1% | 8.2% | |
| Azathioprine | 14.7% | 1.4% | 0.0% | 0.4% | | 0.0% | 1.8% | | 0.0% | 1.2% | 1.6% | |
| Corticosteroids | 91.1% | 82.2% | 97.4% | 94.3% | | 94.4% | 91.7% | | 88.7% | 90.7% | 82.8% | |
| Zenapax | 2.5% | 9.4% | 2.6% | 4.5% | | 0.0% | 7.3% | | 6.5% | 1.2% | 4.1% | |
| Days Post-Tx | | | | | | | | | | | | |
| Average Days | NA | 241 | 83 | 62 | | 254 | 206 | | 205 | 224 | 265 | |
| Post-Tx | | | | | | | | | | | | |

NS = Not significant (P ≧ 0.05)
*From UNOS 2001 data. This percentage represents the number of transplants in which a particular drug was used for maintenance at any point in the year after transplant divided by the number of transplants in 2001, and only accounts for patients with immunosuppressive information.

TABLE 24
Discriminant Algorithm Performance

| | Months | | Biopsy Grade 3A Rejection | | | | Biopsy Grade 0 | | | |
|---|---|---|---|---|---|---|---|---|---|---|
| Sample set | post Tx | Threshold | #Patients | #Samples | #Agree | % Agree | #Patients | #Samples | #Agree | % Agree |
| Training | All | 20 | 29 | 36 | 29* | 80.0%* | 99 | 109 | 64* | 59.0%* |
| Validation | All | 20 | 50 | 62 | 47 | 75.8% | 83 | 122 | 51 | 41.8% |
| Validation | All | 20 | 32 | 37 | 29 | 78.4% | 31 | 36 | 17 | 47.2% |
| unique | | | | | | | | | | |
| Validation | ≦4 | 18.5 | 24 | 31 | 23 | 74.2% | 40 | 62 | 30 | 48.4% |
| Validation | ≦4 | 18.5 | 16 | 17 | 13 | 76.4% | 18 | 21 | 11 | 52.4% |
| unique | | | | | | | | | | |
| Validation | >4 | 26.5 | 26 | 31 | 23 | 74.2% | 43 | 60 | 39 | 65.0% |
| Validation | >4 | 26.5 | 18 | 20 | 16 | 80.0% | 15 | 15 | 11 | 73.3% |
| unique | | | | | | | | | | |
| Validation | >6 | 28 | 19 | 21 | 15 | 71.4% | 38 | 47 | 37 | 78.7% |
| Validation | >6 | 28 | 12 | 12 | 10 | 83.3% | 14 | 14 | 11 | 78.6% |
| unique | | | | | | | | | | |
| Validation | >12 | 30 | 10 | 10 | 8 | 80.0% | 15 | 18 | 14 | 77.8% |
| Validation | >12 | 30 | 5 | 5 | 5 | 100.0% | 7 | 7 | 4 | 57.1% |
| unique | | | | | | | | | | |

*bootstrap estimates

We claim:

1. A method of diagnosing or monitoring the functional status of a transplant in a subject comprising:
a) detecting the expression levels of all genes of a diagnostic gene set in the subject wherein the diagnostic gene set comprises at least one gene from each of at least two gene tables selected from the group consisting of table 1, table 2, table 3, table 4, table 5, table 6, table 7, table 8, table 9, table 10, table 11; table 12, table 13, table 14, table 15 and table 16; and
b) diagnosing or monitoring the functional status of the transplant in the subject based upon the expression levels of the genes in the diagnostic gene set.

2. The method of claim 1 wherein the transplant is selected from the group consisting of cardiac transplant, lung transplant, and renal transplant.

3. The method of claim 1 wherein the expression levels of the genes in the diagnostic gene set are detected using the same method of detection.

4. The method of claim 1 wherein the expression levels are detected by measuring the RNA level of the genes in the diagnostic gene set.

5. The method of claim 4 further comprising isolating the RNA from said subject prior to detecting the RNA level of the genes in the diagnostic gene set.

6. The method of claim 4 wherein said RNA level is detected the method selected from the group comprising PCR and hybridization.

7. The method of claim 6 wherein said RNA level is detected by hybridization to an oligonucleotide.

8. The method of claim 7 wherein the oligonucleotide comprises DNA, RNA, cDNA, PNA, genomic DNA, or a synthetic oligonucleotide.

9. The method of claim 1 wherein the diagnosing or monitoring is performed by applying an algorithm to the expression levels of the genes of the diagnostic gene sets.

10. The method of claim 9 wherein the algorithm is a linear algorithm.

11. The method of claim 9 wherein the algorithm is optimized to assign the subject to one of at least two categories.

12. The method of claim 9 wherein the algorithm is optimized to assign the subject to one of two categories.

13. The method of claim 11 wherein the optimization includes maximization of the separation between the at least two categories.

14. The method of claim 1 wherein the functional status monitored is selected from the group comprising acute rejection, chronic rejection and likelihood of future rejection.

15. The method of claim 1 wherein the diagnostic gene set comprises at least one gene from each of at least three gene tables.

16. The method of claim 1 wherein the diagnostic gene set comprises at least one gene from each of at least five gene tables.

17. The method of claim 1 wherein the diagnostic gene set comprises at least two gene from each of at least two gene tables.

18. A method of generating a probe set for diagnosing or monitoring transplant rejection in a subject comprising:
a) generating a diagnostic gene set by selecting at least one gene from each of at least two gene tables selected from the group consisting of table 1, table 2, table 3, table 4, table 5, table 6, table 7, table 8, table 9, table 10, table 11; table 12, table 13, table 14, table 15 and table 16; and
b) generating a probe set by creating at least one probe that specifically detects the expression level for each gene in the diagnostic gene set.

19. A probe set comprising at least one probe for detection of expression of at least one gene from each of at least two gene tables selected from the group consisting of table 1, table 2, table 3, table 4, table 5, table 6, table 7, table 8, table 9, table 10, table 11; table 12, table 13, table 14, table 15 and table 16.

20. A method of diagnosing or monitoring the functional status of a transplant in a subject comprising:
a) detecting the expression levels of all genes of a diagnostic gene set in a patient wherein the diagnostic gene set comprises at least one gene from each of at least two gene clusters selected from the group consisting of Cell-Surface Mediated Signaling Cluster, Inflammation Cluster, Steroid Responsive Gene Cluster, Early Activation Cluster, Heart Failure Cluster, Hematopoiesis Cluster, Megakaryocytes Cluster, T/B Cell Regulation Cluster, Transcription Control Cluster, T Cell Cluster, Inflammatory Cell Recruitment Cluster, Transcription Factor Related Cluster, Dendritic Cell Maturation Cluster, Cell Activation Cluster, Cytotoxic T Cell Cluster, and Bone Marrow Stromal Cell Migration Cluster; and
b) diagnosing or monitoring the functional status of the transplant in the patient based upon the expression levels of the genes in the diagnostic gene set.
